(19) United States
(12) Patent Application Publication
(10) Pub. No.: US 2025/0295727 A1
(43) Pub. Date: Sep. 25, 2025

(54) STABLE, READY-TO-ADMINISTER AQUEOUS FORMULATIONS OF DALBAVANCIN

(71) Applicant: HIKMA PHARMACEUTICALS USA INC., Berkeley Heights, NJ (US)

(72) Inventors: Sardar M. Jakaria, Bedford, OH (US); James Murtagh, Bedford, OH (US); Gurmukh Chanana, Bedford, OH (US)

(21) Appl. No.: 18/860,166

(22) PCT Filed: Dec. 2, 2022
(86) PCT No.: PCT/US2022/051653

§ 371 Date: Oct. 25, 2024

Related U.S. Application Data
(60) Provisional application No. 63/334,892, filed on Apr. 26, 2022.

(51) Int. Cl. A61K 38/08; A61K 47/02; A61K 47/40

(52) U.S. Cl. CPC A61K 38/08; A61K 47/02; A61K 47/40

(57) ABSTRACT

An aqueous pharmaceutical composition for intravenous administration that includes dalbavancin or a pharmaceutically acceptable salt thereof in a pharmaceutically acceptable buffer having a pH of from about 4.0 to about 8.0. The aqueous composition further includes either or both of a pharmaceutically acceptable divalent salt wherein the divalent salt is distinct from the buffer and/or a pharmaceutically acceptable cyclodextrin. The formulation demonstrates improved long-term stability for storage at room or elevated temperatures. Methods of using the aqueous pharmaceutical composition of dalbavancin to treat skin infections.

This application claims priority to U.S. Provisional Patent Application No. 63/334,892, filed Apr. 25, 2022, hereby incorporated by reference in its entirety.

### FIELD

The invention relates to a heat-stable aqueous formulation comprising dalbavancin as an active ingredient and, in particular, stable, ready-to-administer aqueous formulations for intravenous infusion comprising dalbavancin and a buffer having improved stability profiles. The formulations can further comprise metal ions and/or cyclodextrins for improved stability.

### BACKGROUND

Dalbavancin is a second-generation lipoglycopeptide antibiotic approved for the treatment of patients with acute bacterial skin and skin structure infections caused by certain strains of Gram-positive bacteria, including, e.g., methicillin-resistant Staphylococcus aureus (CLSA) and methicillin-resistant Staphylococcus epidermidis (CLSE) and is currently being evaluated for efficacy and safety in adult subjects with osteomyelitis. In addition, dalbavancin is an emerging treatment option for most of the MRD/XRD microorganisms which are Gram-positive cocci bacteria.

Dalbavancin is marketed under the tradename DALVANCE® in the United States and XYDALBAR in Europe. It is derived from the natural product glycopeptide by amidation of the peptide-carboxy group of amino acid 7 with 3-(dimethylamino)-1-propylamine. The introduction of this substituent increases the potency against staphylococci, particularly coagulase-negative staphylococci. Dalbavancin is a mixture of five closely related active homologs or factors (A₀, A₁, B₀, B₁ and B₂), all of which are bactericidally active against a number of Gram-positive bacteria. The homologs all share the same core structure but differ in the fatty acid side chain of the N-acylaminoglucuronic acid moiety (R1) and/or the presence of an additional methyl group (R2) on the terminal amino group. The B₀ factor is the predominant component of the active material.

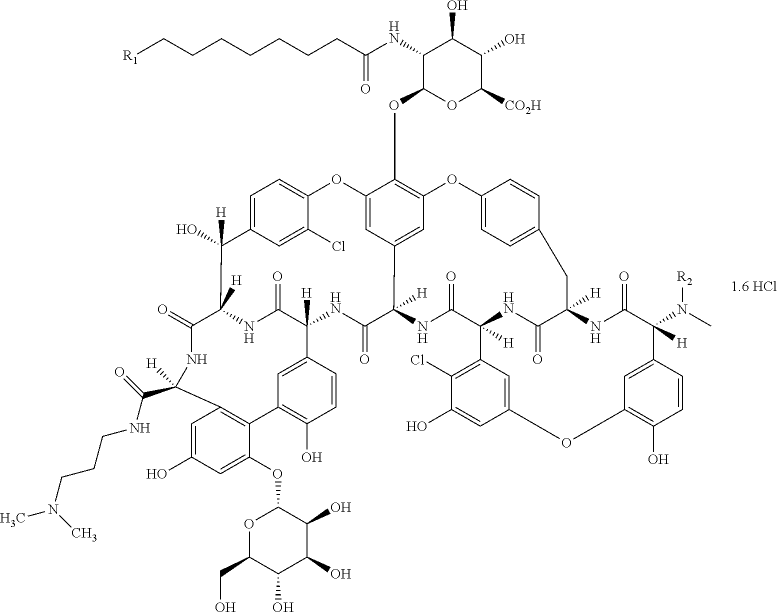

| | | | | Molecular |
|---|---|---|---|---|
| | | | | Weight |
| | | | | (anhydrous |
| Dalbavancin | R1 | R2 | Molecular Formula | free base) |
| A₀ | CH(CH₃)₂ | H | C₈₇H₉₈N₁₀O₂₈Cl₂•1.6 HCl | 1802.7 |
| A₁ | CH₂CH₂CH₃ | H | C₈₇H₉₈N₁₀O₂₈Cl₂•1.6 HCl | 1802.7 |
| B₀ | CH₂CH(CH₃)₂ | H | C₈₇H₉₈N₁₀O₂₈Cl₂•1.6 HCl | 1816.7 |
| B₁ | CH₂CH₂CH₂CH₃ | H | C₈₇H₉₈N₁₀O₂₈Cl₂•1.6 HCl | 1816.7 |
| B₂ | CH₂CH(CH₃)₂ | CH₃ | C₈₇H₉₈N₁₀O₂₈Cl₂•1.6 HCl | 1830.7 |
| MAG | N/A | H | C₈₇H₉₈N₁₀O₂₈Cl₂•1.6 HCl | 1459.27 |
| (Mannosyl | | | | |
| Aglycone) | | | | |

Glycopeptides, particularly dalbavancin, are very unstable in aqueous solution due to the glycosidic linkage [79]. The primary degradant of dalbavancin in aqueous solution is a non-homologous component referred to as Mannosyl Aglycone (MAG). MAG is the result of the hydrolysis of the glycosidic linkage of dalbavancin, resulting in a less bactericidal component which lacks the acylglucoronamine moiety. To minimize such degradation, the marketed product is sold in the US as a vial for reconstitution, the vial comprising the hydrochloride salt of dalbavancin as a lyophilized powder (500 mg of free base), lactose (129 mg) and mannitol (129 mg). The instructions for reconstitution instruct that the product is to be reconstituted with either sterile water for injection, USP, or 5% dextrose injection, USP, followed by dilution only with 5% dextrose injection, USP to a final concentration of 1 mg/mL to 5 mg/mL. According to the labelling instructions, reconstituted vials or diluted intravenous bags may be stored either refrigerated (2 to 8° C.) or at a controlled room temperature (20 to 25° C.). The instructions further indicate that the total time from reconstitution to dilution to administration should not exceed 48 hours. The European XYDALBA product is likewise a single use vial comprising dalbavancin, lactose and mannitol for reconstitution in water and dilution in 5% glucose. The XYDALBA label further indicates that the use of sodium chloride-containing solutions will cause precipitation and should not be used for reconstitution or dilution. Importantly, the XYDALBA label clearly states that the chemical and physical in-use stability for both the reconstituted and diluted solutions has been demonstrated to be 48 hours at or below 25° C.

Long term stability data for dalbavancin was reported in U.S. Pat. No. 8,143,212. Even in lyophilized form, the long-term stability of dalbavancin presents challenges for long term storage. A study of the stability of unformulated lyophilized dalbavancin reported a 2.9% increase in MAG and a proportional 3.6% decrease in dalbavancin's primary factor B₀ following storage for 36 months at 5° C. More extensive degradation of the lyophilized product was observed following storage at 25° C. for 12 months, with an increase of MAG of 10.2% and a corresponding decrease of B₀ of 8.5%. It is likewise established therein that dalbavancin is unstable in aqueous solutions.

Thus, there remains a need for ready-to-use dalbavancin formulations that demonstrate long-term stability across a broad range of temperatures, including temperatures at or above 25° C., particularly for use in areas where the lack of a cold storage is a limiting factor for injectable drug products. The present disclosure provides stable aqueous formulations of dalbavancin having minimal ingredients that exhibit enhanced storage stability as compared to the stated stability and storage limitations of the commercially available product.

### SUMMARY

Described herein are aqueous pharmaceutical compositions for intravenous administration that include dalbavancin or pharmaceutically acceptable salts thereof and one or more of metal cations and/or cyclodextrin in an aqueous buffer. The formulations exhibit improved stability at standard and accelerated storage conditions compared to commercially available dalbavancin formulations, which are available only as lyophilized powders for reconstitution.

In a first aspect, there is disclosed an aqueous pharmaceutical composition for intravenous administration that includes dalbavancin or a pharmaceutically acceptable salt(s) thereof and a pharmaceutically acceptable buffer having a pH of from about 4.0 to about 8.0. The formulation further includes one or more of a pharmaceutically acceptable divalent salt and/or a pharmaceutically acceptable cyclodextrin. The divalent salt is distinct from the buffer. The aqueous pharmaceutical composition according to the first aspect retains about 90% or more of the initial concentration of dalbavancin or pharmaceutically acceptable salt thereof after it has been stored for at least six months at about 5° C.

In an example of the first aspect, the dalbavancin is dalbavancin hydrochloride.

In another example of the first aspect, the composition has a pH of from about 4.0 to about 5.0.

In another example of the first aspect, the buffer is an acetate buffer.

In yet another example of the first aspect, the composition has a pH of about 6.5 to about 7.5.

In another example of the first aspect, the buffer is a phosphate buffer.

In another example of the first aspect, the buffer has a concentration of from about 1 μM to about 20 mM.

In yet another example of the first aspect, the composition includes a divalent salt. The cation of the divalent salt is selected from the group consisting of Ca²⁺, Mg²⁺, Zn²⁺ and mixtures thereof.

In one example of the first aspect, the divalent cation is Ca²⁺.

In one another example of the first aspect, the divalent cation is Mg²⁺.

In one yet another example of the first aspect, the divalent cation is Zn²⁺.

In another example of the first aspect, the counter anion of the divalent salt is Cl⁻.

In another example of the first aspect, the divalent cation is present in an amount of from about 0.1 mM to about 50 mM.

In still another example of the first aspect, the divalent cation is present in an amount of from about 0.1 mM to about 20 mM.

In another example of the first aspect, the formulation comprises the cyclodextrin.

In another example of the first aspect, the cyclodextrin is 2-hydroxypropyl-β-cyclodextrin.

In still another example of the first aspect, the cyclodextrin is present in an amount of from about 0.5 mM to about 200 mM or in an amount of from about 10 mM to about 50 mM.

In yet still another example of the first aspect, the composition comprises both the divalent salt and the cyclodextrin.

In another example of the first aspect, the cation of the divalent salt is selected from the group consisting of Ca²⁺, Mg²⁺, Zn²⁺ and mixtures thereof and the cyclodextrin is 2-hydroxypropyl-β-cyclodextrin.

In yet another example of the first aspect, the divalent salt is present in an amount of from about 1 mM to about 20 mM.

In yet another example of the first aspect, the cyclodextrin is present in an amount of from about 0.5 mM to about 200 mM.

In another example of the first aspect, the pharmaceutical composition further comprises one or more pharmaceutically acceptable excipients.

In yet another example of the first aspect, the one or more pharmaceutically acceptable excipients includes a preservative.

In still another example of the first aspect, the composition excludes a preservative.

In another example of the first aspect, the composition is stable for at least 1 month, at least 2 months, at least 3 months or at least 6 months when stored at about 5° C.

In another example of the first aspect, the composition is stable for at least 1 month, at least 2 months, at least months or at least 6 months when stored at about 25° C. and about 60% relative humidity.

In another example of the first aspect, the composition is stable for at least 1 month when stored at about 40° C. and about 75% relative humidity.

In another example of the first aspect, the composition includes a concentration of dalbavancin or pharmaceutically acceptable salt thereof of between about 0.01 mg/mL and about 50 mg/mL.

In another example of the first aspect, the composition retains about 90% or more of an initial concentration of dalbavancin or pharmaceutically acceptable salt thereof after storage at when stored at about 25° C. and about 60% relative humidity for at least 1 month.

In another example of the first aspect, the composition retains about 94% or more of an initial concentration of dalbavancin or pharmaceutically acceptable salt thereof after storage at when stored at about 25° C. and about 60% relative humidity for at least 1 month.

In another example of the first aspect, the composition retains about 80% or more of an initial concentration of a B₀ component of dalbavancin or pharmaceutically acceptable salt thereof after storage at when stored at about 25° C. and about 60% relative humidity for at least 1 month.

In another example of the first aspect, the composition retains about 85% or more of an initial concentration of a B₀ component of dalbavancin or pharmaceutically acceptable salt thereof after storage at when stored at about 25° C. and about 60% relative humidity for at least 1 month.

In another example of the first aspect, the composition retains about 90% or more of an initial concentration of dalbavancin or pharmaceutically acceptable salt thereof after storage at when stored at about 40° C. and about 75% relative humidity for at least 1 month.

In another example of the first aspect, the composition the composition retains about 85% or more of an initial concentration of a B₀ component of dalbavancin or pharmaceutically acceptable salt thereof after storage at when stored at about 40° C. and about 75% relative humidity for at least 1 month.

In yet another example of the first aspect, the composition contains not more than about 7.0% of a MAG impurity after storage at about 5° C. for at least 1 month.

In yet another example of the first aspect, the composition contains not more than about 7.0% of a MAG impurity after storage at about 5° C. for at least 6 months.

In yet another example of the first aspect, the composition contains not more than about 5.0% of a MAG impurity after storage at about 5° C. for at least 1 month.

In yet another example of the first aspect, the composition contains not more than about 5.0% of a MAG impurity after storage at about 5° C. for at least 6 months.

In yet another example of the first aspect, the composition contains not more than about 7.0% of a MAG impurity after storage at about 25° C. and about 60% relative humidity for at least 1 month.

In yet another example of the first aspect, the composition contains not more than about 7.0% of a MAG impurity after storage at about 25° C. and about 60% relative humidity for at least 3 months.

In yet another example of the first aspect, the composition contains not more than about 5.0% of a MAG impurity after storage at about 25° C. and about 60% relative humidity for at least 1 month.

In yet another example of the first aspect, the composition contains not more than about 5.0% of a MAG impurity after storage at about 25° C. and about 60% relative humidity for at least 3 months.

In yet another example of the first aspect, the composition contains not more than about 7.0% of a MAG impurity after storage at about 40° C. and about 75% relative humidity for at least 1 month.

In yet another example of the first aspect, the composition contains not more than about 5.0% of a MAG impurity after storage at about 40° C. and about 75% relative humidity for at least 1 month.

In another example of the first aspect, the composition is stored in a glass vial.

In a second aspect of the disclosure is an aqueous pharmaceutical composition for intravenous administration. The composition consists essentially of dalbavancin or a pharmaceutically acceptable salt thereof, a pharmaceutically acceptable buffer and one or more of a pharmaceutically acceptable divalent salt and/or a pharmaceutically acceptable cyclodextrin. The divalent salt is distinct from the buffer. The dalbavancin is present in an amount of between about 1.0 mg/mL and about 50.0 mg/mL. The composition has a pH of from about 4.0 to about 8.0. The pharmaceutical composition of the second aspect retains about 90% or more of the initial concentration of dalbavancin or pharmaceutically acceptable salt thereof after storage for at least one month when stored at about 5° C.

In a third aspect of the disclosure is an aqueous pharmaceutical composition for intravenous administration. The composition consists essentially of dalbavancin or a pharmaceutically acceptable salt thereof, an acetate buffer and one or more of a pharmaceutically acceptable divalent salt and/or a pharmaceutically acceptable cyclodextrin. The divalent salt is distinct from the buffer. The dalbavancin is present in an amount of between about 1.0 mg/mL and about 30.0 mg/mL. The composition has a pH of from about 4.0 to about 5.0. The pharmaceutical composition of the third aspect retains about 90% or more of the initial concentration of dalbavancin or pharmaceutically acceptable salt thereof after storage for at least six months when stored at about 5° C.

In an example of the third aspect, the pharmaceutical composition includes both the divalent salt and the cyclodextrin.

In another example of the third aspect, the cation of the divalent salt is selected from the group consisting of Ca²⁺, Mg²⁺, Zn²⁺ and mixtures thereof and the cyclodextrin is 2-hydroxypropyl-β-cyclodextrin.

In another example of the third aspect, the divalent salt is present in an amount of from about 1 mM to about 20 mM.

In another example of the third aspect, the cyclodextrin is present in an amount of from about 0.5 mM to about 200 mM.

In a fourth aspect of the disclosure is an aqueous pharmaceutical composition for intravenous administration. The composition consists essentially of dalbavancin or a pharmaceutically acceptable salt thereof, a phosphate buffer and one or more of a pharmaceutically acceptable divalent salt and/or a pharmaceutically acceptable cyclodextrin. The divalent salt is distinct from the buffer. The dalbavancin is present in an amount of between about 1.0 mg/mL and about 50.0 mg/mL. The composition has a pH of from about 6.5 to about 7.5. The pharmaceutical composition of the fourth aspect retains about 90% or more of the initial concentration of dalbavancin or pharmaceutically acceptable salt thereof after storage for at least one month when stored at about 5° C.

In an example of the fourth aspect, the pharmaceutical composition includes both the divalent salt and the cyclodextrin.

In another example of the fourth aspect, the cation of the divalent salt is selected from the group consisting of Ca²⁺, Mg²⁺, Zn²⁺ and mixtures thereof and the cyclodextrin is 2-hydroxypropyl-β-cyclodextrin.

In another example of the fourth aspect, the divalent salt is present in an amount of from about 1 mM to about 20 mM.

In another example of the fourth aspect, the cyclodextrin is present in an amount of from about 0.5 mM to about 200 mM.

In an example of any of the previous aspects is a method of administering the pharmaceutical composition to a mammal in need thereof.

In a fifth aspect of the disclosure is a method treating a Gram-positive infection in a patient in need thereof. The method includes the steps of providing an aqueous pharmaceutical composition; and intravenously administering the pharmaceutical composition to the patient. The pharmaceutical composition comprises between about 1.0 mg/mL and about 30.0 mg/mL dalbavancin or a pharmaceutically acceptable salt thereof; a pharmaceutically acceptable buffer having a pH of from about 4.0 to about 8.0; and one or more of a pharmaceutically acceptable divalent salt and/or a pharmaceutically acceptable cyclodextrin. When the pharmaceutically acceptable divalent salt is present, the divalent salt is distinct from the buffer. After storage at about 5° C. for at least 6 months, the pharmaceutical composition retains at least 90% of the initial dalbavancin concentration.

In an example of the fifth aspect, the Gram-positive infection is a skin infection.

### DETAILED DESCRIPTION

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the endpoints of each of the ranges are significant both in relation to the other endpoint, and independently of the other endpoint and that ranges include both of the endpoints as well as each of the discreet values therein.

The terms “substantial,” “substantially,” and variations thereof as used herein are intended to note that a described feature is equal or approximately equal to a value or description. Moreover, “substantially” is intended to denote that two values are equal or approximately equal. In some embodiments, “substantially” may denote values within about 10% of each other, for example within about 5% of each other, or within about 2% of each other.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue

As used herein, the term “consisting essentially of” is intended to indicate an exclusion of pharmaceutical excipients that materially affect the basic and novel characteristics of the formulation. More specifically, as used herein, a pharmaceutical formulation “consisting essentially of” excludes pharmaceutical ingredients or excipients other than those specified along with any impurities or degradation products that may result from the ingredients included in the disclosed formulation.

The present disclosure is directed to stable aqueous formulations for intravenous administration that include dalbavancin, or a pharmaceutically acceptable salt thereof, in a buffer, and further comprising either or both of metal ions, specifically divalent cations, and a cyclodextrin. The buffered dalbavancin formulations of the current disclosure demonstrate the high stability in solution for extended storage or extended storage at elevated temperatures.

The formulations of dalbavancin of the present disclosure can be for intravenous or parenteral administration. In some examples, the formulations are ready-to-use or ready to administer formulations such as a liquid stored in a pharmaceutically suitable container, for example, a glass vial or plastic intravenous bag. A ready-to-use or ready-to-administer formulation is a sterile, liquid injectable formulation not requiring reconstitution before use such that the formulation can be further diluted if present as a concentrated solution, or directly administered. For example, a ready-to-administer formulation can be included at the required concentration and volume in the final container such as a syringe or injector. A ready-to-use preparation can be at the required concentration and a volume in a container that may be transferred to a final administration device such as a syringe or infusion bag for administration to a patient. Diluents can include, for instance, fluids suitable for parenteral administration such as sodium chloride or dextrose solutions. In one example, the pharmaceutical composition is a ready-to-use preparation.

The pharmaceutical compositions provided herein may be formulated for single or multiple dosage administration. Multiple dosage formulations may include an antimicrobial agent at bacteriostatic or fungistatic concentrations. All formulations must be sterile, as known and practiced in the art.

The liquid formulations of the present disclosure are stable or exhibit stability when stored, which includes formulation properties that may be affected by storage conditions, for example, active ingredient strength or concentration, impurities (e.g., individual components and total), visual appearance characteristics (e.g., color, clarity, cloudy, haze, precipitates, etc.) and viscosity. Storage conditions that may affect stability can include, for example, storage temperature, humidity (e.g., relative), light exposure and storage time period.

In one or more embodiments, stability can include the amount of total impurities, inclusive of degradation products, that are generated after formation of the dalbavancin formulation for a specified period of time at specified storage conditions (e.g., temperature, humidity) minus the initial total impurities as measured following formation (i.e. the baseline or initial impurity measurement). As used herein, “stability” or a “stable formulation” refers to a formulation demonstrating one or more of the following characteristics upon inspection. The formulations can be examined visually. Stable formulations are essentially clear solutions that are free or essentially free from visible signs of contamination from any foreign materials and/or particulate matter. As used herein, essentially clear formulations are formulations that are clear, colorless and/or slightly hazy. Formulations that are essentially clear are considered to be stable over the period tested. The formulations can be tested for pH stability over time. By this measure, formulations are deemed to be stable when the pH varies less than ±0.5 pH units over the period of storage. The formulations can also be measured for chromatographic (HPLC) purity using relative peak areas determined from a standard set of run parameters. First, following a period of storage, the relative value of dalbavancin remaining in the formulation as compared to a known amount of dalbavancin (e.g. a dalbavancin standard) can be measured (referred to as “Assay %”). Stable samples according to the Assay % should demonstrate 90.0 to 110.0% (±10.0%) dalbavancin remaining relative to standard as a measure of peak area. A second chromatographic test (referred to as “Chromatographic purity (%)”) measures the peak area of the dalbavancin peak relative to the total peak chromatographic peak area. By this measurement, stable formulations retain 90.0% or greater of the original dalbavancin concentration by chromatographic determination and/or 80.0% or greater of the B₀ component of dalbavancin following extended storage. As dalbavancin has multiple components, in both of the chromatography assessments, it is total dalbavancin content that is of interest rather than any particular form. In other words, a dalbavancin formulation according to the present disclosure will be considered “stable” if the total dalbavancin content is, e.g. 90.0% or greater following storage regardless of the amounts of the individual components present after storage. In other embodiments, a dalbavancin formulation according to the present disclosure will be considered “stable” if the B₀ component is 80.0% or greater, or preferably 85.0% or greater following storage regardless of the amounts of the other components present after storage. In some aspects, stable formulations are those formulations demonstrating visual stability over the storage period. In some examples, stable formulations are those formulations that maintain pH stability over the storage period. In other examples, stable formulations are those formulations that demonstrate stability by one or more chromatographic assay(s) over the storage period. In yet other examples, stable formulations demonstrate two or three or all of visual stability, pH stability, and chromatographic stability over the storage period.

In one or more embodiments, a liquid dalbavancin formulation includes a formulation that retains about 90.0% or more, about 91.0% or more, about 92.0% or more, about 93.0% or more, about 94.0% or more, about 95.0% or more, about 96.0% or more, about 97.0% or more, about 98.0% or more, or about 99.0% or more of the initial concentration of dalbavancin (or it total components) or pharmaceutically acceptable salt thereof in the formulation after storage under standard (e.g., 2-8° C., or “refrigerated” conditions), room temperature (e.g., about 15° C. to about 30° C., including about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., or any range in between), or accelerated conditions (e.g., 30° C./65% Relative Humidity (RH), 40° C./75% RH, etc.). The initial concentration of dalbavancin or a pharmaceutically acceptable salt can be measured shortly after formation and filling of the formulation into a pharmaceutically acceptable container (e.g., a vial) prior to storage. For example, filling of the formulation can be within 1-24 hours of formation of the formulation. In one or more embodiments, a liquid dalbavancin formulation includes a formulation that retains about 80.0% or more, about 81.0% or more, about 82.0% or more, about 83.0% or more, about 84.0% or more, about 85.0% or more, about 86.0% or more, about 87.0% or more, about 88.0% or more, or about 89.0% or more, about 90.0% or more, about 91.0% or more, about 92.0% or more, about 93.0% or more, about 94.0% or more, about 95.0% or more, about 96.0% or more, about 97.0% or more, about 98.0% or more, or about 99.0% or more of the B₀ component of dalbavancin or pharmaceutically acceptable salt thereof in the formulation after storage under standard (e.g., 2-8° C., or “refrigerated” conditions), room temperature (e.g., about 15° C. to about 30° C., including about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., or any range in between), or accelerated conditions (e.g., 30° C./65% Relative Humidity (RH), 40° C./75% RH, etc.). The initial and final concentrations of the B₀ component can be measured shortly after formation and filling of the formulation into a pharmaceutically acceptable container (e.g., a vial) prior to storage. For example, filling of the formulation can be within 1-24 hours of formation of the formulation. In one or more embodiments, a stable dalbavancin formulation includes a formulation that contains about 0.5% or less, about 1.0% or less, about 1.5% or less, about 2.0% or less, about 2.5% or less, about 3.0% or less, about 3.5% or less about 4.0% or less, about 4.5% or less, about 5.0% or less of an individual impurity, about 5.5% or less of an individual impurity, or about 6.0% or less of an individual impurity (e.g., a degradation impurity such as MAG) formed after formation of the formulation and present after storage under standard, room temperature, or accelerated conditions (e.g., about 2-8° C., 25° C./60% RH, 30° C./65% RH, 40° C./75% RH or 50° C.) for about 1 week, about 2 weeks, about 3 weeks, about 4 weeks (1 month), about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, or about 18 or more months. Any measured individual impurity for purposes of measuring stability of a formulation herein does not include any impurity present in any ingredient prior to formation of the dalbavancin formulation. That is, as used herein, an impurity or impurities in an invented formulation refers to any impurity, including a degradation product, formed after formation of the formulations. In a preferred embodiment according to the disclosure, the dalbavancin formulation comprises less than about 6.0% (chromatographic area) of MAG, less than about 1.0% of other known impurities and less than about 0.5% of other unknown impurities

In one or more embodiments, a liquid dalbavancin formulation includes a formulation that is stable for about 3 months or more, about 6 months or more, about 9 months or more, about 12 months or more, or about 18 months or more when stored at an accelerated temperature of about 25° C./60% RH, 30° C./65% RH or 40° C./75% RH. In one or more embodiments, a liquid dalbavancin formulation includes a formulation that is stable for about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months or more when stored at about room temperature (i.e. 20-25° C.).

The formulations of the present disclosure contain, as the active ingredient, dalbavancin or any pharmaceutically acceptable salt thereof. In some embodiments, the formulations preferably contain dalbavancin or any pharmaceutically acceptable salt thereof as the sole active ingredient characterized in that no other active ingredients are present in the formulation. In one example, the formulation contains dalbavancin hydrochloride.

The pharmaceutical composition (or formulation) may be administered intravenously in a therapeutically effective amount of dalbavancin. A therapeutically effective amount of dalbavancin can be present in an amount of, for example, about 100 mg, about 200 mg, about 300 mg, about 400 mg, about 500 mg, about 600 mg, about 700 mg, about 800 g, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg or about 3000 mg. In some embodiments, the dalbavancin formulations of the present disclosure can be administered in a single dose of, for example, 1500 mg. In some embodiments, the dalbavancin formulations can be administered in multiple doses, such as, for example, a 1000 mg dose followed by a 500 mg dose.

In addition to the active pharmaceutical ingredient, formulations of the present disclosure include a buffer or buffering agent as a diluent. As used herein, the term “buffer” includes both a pre-made buffer or a buffer made in situ by the addition of a pair of buffer salts, e.g. a weak acid or weak base plus a salt of the weak acid or weak base, respectively, and any pH adjusting agents as needed. Suitable buffers or buffering agents include, e.g., acetate buffer having a pH of between about 3.0 and 6.0, preferably between 4.0 and 5.0, most preferably about 4.5 and phosphate buffer having a pH of between about 6.0 and 8.0, preferably between 6.5 and 7.5, most preferably about 7.0. The concentration of the buffer can range from about 0.1 mM to about 100 mM, for example, about 0.1 mM, about 0.5 mM, about 1.0 mM, about 1.5 mM, about 2.0 mM, about 2.5 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM, about 10.0 mM, about 10.5 mM, about 11.0 mM, about 11.5 mM, about 12.0 mM, about 12.5 mM, about 13.0 mM, about 13.5 mM, about 14.0 mM, about 14.5 mM, about 15.0 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM.

The formulations can be supplied or stored in any suitable volume for intravenous administration or for dilution prior to intravenous administration. In one or more embodiments, the formulation volume (e.g., amount of liquid in a storage container) is about 1 mL, about 2 mL, about 3 mL, about 4 mL, about 5 mL, about 10 mL, about 20 mL, about 30 mL, about 40 mL, about 50 mL, about 100 mL, about 500 mL or about 1000 mL or more. For example, the formulation volume can be about 0.1 mL to about 50 mL, about 0.5 mL to about 5 mL, or about 1 mL. In some embodiments, the formulation volume is about 1 mL, about 2 mL, about 3 mL, or about 5 mL. In one example, the formulation volume is about 25 mL to about 75 mL. In another example, the formulation volume is about 500 to about 2000 mL, about 1000 mL, or about 1500 mL. Appropriate-sized containers for storing formulation volumes can be determined by one of ordinary skill in the art.

The formulations can be stored in or supplied in any suitable container. For example, the formulation can be in a container that includes, but is not limited to, a vial, ampoule, bag (IV bag), bottle, or syringe (e.g., pre-filled syringe or component of an auto-injector). The container can be made of any suitable material, for instance, glass, plastic, or rubber. Prior to filling the formulation in a container, the container is preferably sterile and has been subjected to a sterilization process prior to filling with the sterile formulations of the invention. Containers are sealed as typical in the industry, for example, with the use of a lid, cap, closure, stopper and the like. The containers also can be coated or treated with one or more components to reduce reaction with ingredients of the formulation. For example, a container surface in contact with the formulation can be coated with silicon or a vial with a treated inner surface for storing the formulation can be used. To shield the formulation from exposure to light, a container can optionally be opaque or tinted with a color, and preferably stored in a box for transport or shelving.

The formulations can further include a pH adjuster, for example, an acid or a base. The pH adjuster serves to aid in adjusting the pH of the aqueous formulation. In one or more embodiments, the pH adjuster includes hydrochloric acid. In one or more embodiments, the pH adjuster includes sodium hydroxide. In some embodiments, an acid and base may both be used as pH adjusting agents. The concentration of the pH adjuster can be any concentration suitable for adjusting the pH, such as, for example, 1 N acid or base. The formulation can have any suitable acidic or neutral pH. In an example, the formulation can have a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In one or more embodiments, the formulation can have a pH in the range of about 4.0 to about 5.0, about 4.1 to about 5.0, about 4.2 to about 5.0, about 4.3 to about 5.0, about 4.4 to about 5.0, about 4.5 to about 5.0 about 4.6 to about 5.0, about 4.7 to about 5.0, about 4.8 to about 5.0 or about 4.9 to about 5.0. In another example, the pH of the formulation is about 4.0, about 4.1, about 4.2, about 4.3, about 4.4, about 4.5, about 4.6, about 4.7, about 4.8, about 4.9, or about 5.0. In another aspect of the present disclosure, the pH of the formulation is about 4.5±0.5 pH units, about 4.5±0.4 pH units, about 4.5±0.3 pH units, about 4.5±0.2 pH units or about 4.5±0.1 pH units. In another aspect, the pH of the formulation is about 4.5. In one or more embodiments, the formulation can have a pH in the range of about 6.5 to about 7.5, about 6.6 to about 7.5, about 6.7 to about 7.5, about 6.8 to about 7.5, about 6.9 to about 7.5, about 7.0 to about 7.5, about 7.1 to about 7.5, about 7.2 to about 7.5, about 7.3 to about 7.5 or about 7.4 to about 7.5. In another example, the pH of the formulation is about 6.5, about 6.6, about 6.7, about 6.8, about 6.9, about 7.0, about 7.1, about 7.2, about 7.3, about 7.4 or about 7.5. In another aspect of the present disclosure, the pH of the formulation is about 7.0±0.5 pH units, about 7.0±0.4 pH units, about 7.0±0.3 pH units, about 7.0±0.2 pH units or about 7.0±0.1 pH units. In another aspect of the disclosure, the pH of the formulation is about 7.0.

The formulations of the present application may further include metal cations. Without being bound by theory, the metal cations are believed to improve the arrangement of the hydrophobic groups of the peptide to minimize their interaction with water. According to the present disclosure, metal cations are added in the form of an ionic salt, e.g. NaCl, KCl, or CaCl₂). Suitable metal cations for use with the present formulation include monovalent cations, divalent cations and trivalent cations. According to one aspect, the cations for use with the disclose formulations are divalent cations. Suitable divalent cations include any pharmaceutically acceptable divalent cation such as Ca²⁺, Co²⁺, Cu²⁺, Mg²⁺, Mn²⁺, Ni²⁺, Zn²⁺, or mixtures thereof. In one example, the divalent cation is Ca²⁺. In another example, the divalent cation is Mg²⁺. In another example, the divalent cation is Zn²⁺. The counterion may be any pharmaceutically acceptable anion. One exemplary example of a counter anion is Cl⁻.

Pharmaceutically acceptable cations can be present in amounts of from about 0.1 mM to about 50 mM. For example, the pharmaceutically acceptable cation may be present in an amount of about 0.1 mM, 0.5 mM, 1.0 mM, 5 mM, 10 mM, 15 mM, 20 mM, 25 mM, 30 mM, 35 mM, 40 mM, 45 mM or 50 mM. In one embodiment of the disclosure, the pharmaceutically acceptable cation is present in an amount of from about 1.0 mM to about 20.0 mM, about 5.0 mM to about 15.0 mM or about 10 mM.

According to the disclosure, the dalbavancin formulations may further include a cyclodextrin (CD). CDs are cyclic oligosaccharides that have been used as pharmaceutical adjuvants for many years. Without being bound by theory, it is believed that CDs can interact with appropriately-sized drug molecules to form an inclusion complex for improved aqueous solubility, chemical stability and bioavailability of drugs. Although the use of CDs for enhanced performance is usually limited by the small cavity size, it has been surprisingly found that despite its large size, dalbavancin formulations in aqueous buffers are better stabilized, particularly at higher temperatures, when the formulation comprises a CD. Suitable CDs for use with the disclosed formulations include any pharmaceutically acceptable CD, such as natural CDs (e.g. αCD, βCD, γCD) or derivatized cyclodextrins (e.g. Me-MeβCD, MeγCD, hydroxypropyl-βCD, hydroxypropyl-γCD, sulphobutylether-β-CD, etc.). According to one aspect of the disclosure, the CD for use with the dalbavancin formulations provided herein is 2-hydroxypropyl-β-cyclodextrin (HPβCD).

Cyclodextrins according to the present disclosure can be present in any pharmaceutically acceptable amounts. In one example, the cyclodextrin may be present in an amount of from 0.1 mM to 1000 mM. In one example, the cyclodextrin may be present in an amount of about 0.5 mM, 1.0 mM, 10 mM, 20 mM, 20 mM, 40 mM, 50 mM, 60 mM, 70 mM, 80 mM, 90 mM, 100 mM, 150 mM, 200 mM, 250 mM, 300 mM, 400 mM, 500 mM, or 1000 mM. In one embodiment of the disclosure, the cyclodextrin is present in any amount of from about 0.5 mM to about 200 mM.

As demonstrated in the present disclosure, the inclusion of HPβCD, particularly in combination with divalent cations, preferably Ca²⁺ or Mg²⁺, more preferably Ca²⁺, greatly, and unexpectedly, stabilizes dalbavancin. The effect of HPβCD is demonstrated particularly at higher temperatures, such as 25° C. or 40° C. Without wishing to be bound by theory, the results strongly suggest that 2HPβCD forms a complex with the hydrophobic glycone tail of dalbavancin, suppressing hydrolysis of the glycosidic bond and protecting dalbavancin in solution.

Optional ingredients for formulating liquid formulations, such as diluents, salts, tonicity agents, antioxidants, and preservatives, can be provided to the formulation at any stage in its preparation.

The formulations of the present disclosure are suitable for intravenous administration, for example, to a mammal to treat or prevent a disease or condition. Preferably, the mammal is a human. The disease or condition is treatable by the administration of dalbavancin or a pharmaceutically acceptable salt thereof. In an example, the pharmaceutical composition is administered to infections. In another example the infection is a skin or skin structure infection. In another example, the infection is caused by one or more strain of Gram-positive bacteria.

In order to demonstrate the practice of the present invention, the following examples have been prepared and tested. The examples should not, however, be viewed as limiting the scope of the invention. The claims will serve to define the invention.

### EXAMPLES

### Example 1—Stability of Dalbavancin Formulations in Acetate Buffer with and without Metal Ions

This example demonstrates the stability of formulations comprising dalbavancin hydrochloride in acetate buffer. Dalbavancin formulations were made to include 1 mg/mL, 5 mg/mL or 20 mg/mL dalbavancin in 10 mM acetate buffer. Divalent cations (Ca²⁺, Mg²⁺ and Zn²⁺) were added in the form of calcium chloride, magnesium chloride or zinc chloride salts, respectively. The pH of the formulations was adjusted to 4.5 with 0.1 N HCl and/or 0.1 N NaOH. The formulations were stored in a 5 cc blow back, clear, Type I glass vials with 13 mm, 4432/50 Grey West (Teflon 2 coating) Westar Silicone stoppers. The filter membrane was a Millipore Durapore PVDF disk membrane with a 0.22 μm pore size and 47 mm diameter. Flush volumes were applied to compensate for peptide surface adsorptions. The fill volume was 1 mL. All vials were stored in upright positions under the storage conditions set forth in the examples below.

After fixed periods of storage time (1 month, 2 months, 3 months and 6 months), the stability of the formulations was assessed in several different ways. First, vials were visually inspected ensure they remained as clear, substantially colorless solutions free of particulate matter. As used herein, “cl” indicates that the formulation appearance was a clear, colorless solution that was free of particulate matter. As used herein, “co” indicates that the formulation was a colorless solution. As used herein, “y” indicates that the formulation had a yellow, or slightly yellow coloring. As used herein, “p” indicates that the formulation had a pink, or slightly pink coloring. As used herein, “h” and “sh” indicates that the formulation had an appearance that was hazy or slightly hazy, respectively. Hazy solutions would typically indicate that some precipitation was present but perhaps that the particle sizes not individually visible to the human eye. As used herein, “ppt” indicates that visible precipitate is present in the formulation solution. Second, the pH of each formulation sample was measured. Stable pH values were deemed to be those that varied less than ±0.5 pH units, preferably less than ±0.4 pH units, less than ±0.3 pH units, less than ±0.2 pH units, or less than ±0.1 pH units, over the period of storage. Third, the formulations were tested for chromatographic (HPLC) purity in two different manners. The chromatographic assay (“Assay %” values indicated in the tables below) measured the relative value of dalbavancin remaining in the formulation as compared to a known amount of dalbavancin HCl reference standard. Stable samples as measured demonstrated 90.0 to 110.0% (±10%) dalbavancin remaining relative to standard as a measure of peak area of dalbavancin compared to the peak area of a known dalbavancin standard. A second chromatographic test (“Chromatographic purity (%)”) monitored the purity of dalbavancin and its known impurities by HPLC. Stable samples were measured to be at least 90.0% dalbavancin (sum total of A₀+A₁+B₀+B₁+B₂), preferably greater than 95% dalbavancin. To be stable, these tests further required that less than 6% (chromatographic area) 5% MAG impurity, preferably less than 5% MAG, preferably less than 4% MAG, less than 3% MAG, less than 2% MAG or, most preferably, less than 1% MAG impurity. The best performing formulations (i.e. most stable) were those that were stable upon visual inspection, pH measurement and by one or both chromatographic test(s), and further demonstrated low levels of MAG impurity.

The HPLC conditions (Agilent 1200 or equivalent) for each of the chromatographic assays were as follows: reverse phase column: Welch Ultisil, C18, 4.6 mm×250 mm, 5 μm, P/N: 00201-31043; Mobile phase A: 25 mM sodium dihydrogen phosphate buffer, pH 6.0: Acetonitrile (90:10); Mobile Phase B: 25 mM sodium dihydrogen phosphate buffer, pH 6.0: Acetonitrile (30:70); Column temperature: 50° C.; Flow rate: 1.0 mL/min; Injection volume: 10 μL; Autosampler temperature: 5° C.; Detection wavelength: 280 nm (assay detection experiments) 220 nm (related substances experiments); Separation mode: gradient; Gradient program:

| Time (min) | % Mobile phase A | % Mobile phase B |
|---|---|---|
| 0 | 80 | 20 |
| 7 | 67 | 33 |
| 40 | 67 | 33 |
| 55 | 55 | 45 |
| 60 | 10 | 90 |
| 67 | 10 | 90 |
| 68 | 80 | 20 |
| 80 | 80 | 20 |

Using the above conditions, the retention time (RT) of dalbavancin B₀ (the main component) is approximately 26 min. The RT of the associated dalbavancin components are approximately 16 min for A₀ (relative retention time (RRT) to B₀ 0.58), approximately 17 min for A₁ (RRT 0.63), approximately 27 min for B₁ (RRT 1.08) and approximately 29 min for B₂ (RRT 1.17).

Table 1 below sets forth the stability results of dalbavancin formulations in acetate buffer prepared as described above. The formulations were stored at 5±3° C. for 1 month, 2 months, 3 months and 6 months.

TABLE 1
Stability of 20 mg/mL dalbavancin formulations in 10 mM acetate buffer,
pH 4.5 following extended storage at 5 ± 3° C.

| | | | 1 | 2 | 3 | 6 |
|---|---|---|---|---|---|---|
| | Testing | T₀ | month | months | months | months |
| buffer only | Appearance | cl, co | cl, co | cl, co | cl, co | cl, co |
| | pH | 4.46 | 4.46 | 4.37 | 4.47 | 4.50 |
| | Assay % | 102.8 | 100.4 | 101.3 | 98.0 | 100.2 |
| | Chromatographic purity (%) | 97.97 | 98.16 | 98.66 | 98.63 | 98.56 |
| +2 mM Ca²⁺ | Appearance | cl, co | cl, co | cl, co | cl, co | cl, co |
| | pH | 4.41 | 4.46 | 4.48 | 4.48 | 4.49 |
| | Assay % | 101.1 | 102.2 | 102.3 | 99.2 | 100.3 |
| | Chromatographic purity (%) | 97.94 | 98.15 | 98.62 | 98.63 | 98.56 |
| +10 mM Ca²⁺ | Appearance | cl, co | cl, co | cl, co | cl, co | cl, co |
| | pH | 4.40 | 4.45 | 4.48 | 4.47 | 4.47 |
| | Assay % | 104.2 | 101.4 | 101.5 | 97.6 | 100.6 |
| | Chromatographic purity (%) | 97.95 | 98.27 | 98.64 | 98.62 | 98.55 |
| +2 mM Mg²⁺ | Appearance | cl, co | cl, co | cl, co | cl, co | cl, co |
| | pH | 4.45 | 4.47 | 4.48 | 4.46 | 4.51 |
| | Assay % | 101.8 | 101.2 | 102.2 | 96.6 | 100.8 |
| | Chromatographic purity (%) | 97.96 | 98.16 | 98.68 | 98.64 | 98.56 |
| +2 mM Zn²⁺ | Appearance | cl, co | cl, co | cl, co | cl, co | cl, co |
| | pH | 4.41 | 4.44 | 4.51 | 4.46 | 4.49 |
| | Assay % | 101.7 | 100.8 | 101.7 | 96.3 | 101.3 |
| | Chromatographic purity (%) | 97.94 | 98.22 | 98.69 | 98.62 | 98.56 |

Solution appearance (cl = clear; co = colorless; y = slight yellow color; p = slight pink color; sh = slightly hazy; h = hazy; ppt = precipitated)

As seen from Table 1, 20 mg/mL dalbavancin formulations in acetate buffer were all stable by appearance, pH and by both chromatographic tests for at least 6 months when stored at 5±3° C. All of the tested samples demonstrated less than 2% sample degradation (greater than 98% sample purity by both the assay % and the chromatographic purity testing) following 6 months of storage.

Table 2 below sets forth the analysis of component distribution and degradation products that were identified from the HPLC assay experiments for 20 mg/mL dalbavancin formulations in 10 mM acetate buffer, pH 4.5 initially and following extended storage at 5±3° C. (5° C.) for 1 month, 2 months, 3 months, and 6 months. The percent component distribution was measured by HPLC for the dalbavancin homologs A₀, A₁, B₀, B₁, and B₂ as provided in the table. The amount of degradation products of dalbavancin was also measured for MAG (RT=approximately 4 min). Other impurities include DB-R2 (RT=approximately 30 min, RRT 0.78), DB-R6 (RT=approximately 33 min, RRT=1.26) and any other unidentified (or unknown) impurities. As used in the tables below, “--” indicates that the component was not detected.

TABLE 2
Chromatographic purity analysis of stability/purity of 20 mg/mL
dalbavancin formulations in acetate buffer, pH 4.5 following
extended storage at 5° C. for t = 0, 1, 2, 3 and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| Buffer only | t = 0 | 4.1 | 92.5 | 6.1 | 1.19 | 0.84 |
| | t = 1 mo | 4.0 | 90.1 | 6.2 | 1.27 | 0.57 |
| | t = 2 mo | 4.0 | 88.8 | 5.9 | 1.34 | — |
| | t = 3 mo | 3.9 | 88.6 | 6.1 | 1.37 | — |
| | t = 6 mo | 3.9 | 88.1 | 6.5 | 1.44 | — |
| +2 mM Ca²⁺ | t = 0 | 4.1 | 90.9 | 6.0 | 1.20 | 0.86 |
| | t = 1 mo | 4.2 | 92.1 | 5.9 | 1.27 | 0.58 |
| | t = 2 mo | 4.0 | 88.8 | 5.8 | 1.36 | — |
| | t = 3 mo | 3.9 | 88.7 | 6.1 | 1.37 | — |
| | t = 6 mo | 3.9 | 88.3 | 6.2 | 1.44 | — |
| +10 mM Ca²⁺ | t = 0 | 4.2 | 93.8 | 6.2 | 1.21 | 0.84 |
| | t = 1 mo | 4.1 | 91.1 | 6.3 | 1.31 | 0.42 |
| | t = 2 mo | 4.0 | 88.6 | 6.0 | 1.36 | — |
| | t = 3 mo | 3.9 | 88.6 | 6.1 | 1.38 | — |
| | t = 6 mo | 3.9 | 88.0 | 6.5 | 1.45 | — |
| +2 mM Mg²⁺ | t = 0 | 4.1 | 91.7 | 6.0 | 1.20 | 0.84 |
| | t = 1 mo | 4.0 | 91.2 | 6.1 | 1.27 | 0.57 |
| | t = 2 mo | 4.0 | 88.6 | 6.1 | 1.32 | — |
| | t = 3 mo | 3.9 | 88.6 | 6.1 | 1.36 | — |
| | t = 6 mo | 3.9 | 88.0 | 6.5 | 1.44 | — |
| +2 mM Zn²⁺ | t = 0 | 4.1 | 91.5 | 6.1 | 1.21 | 0.85 |
| | t = 1 mo | 4.1 | 91.9 | 6.1 | 1.26 | 0.52 |
| | t = 2 mo | 4.0 | 89.3 | 5.4 | 1.31 | — |
| | t = 3 mo | 3.9 | 88.6 | 6.1 | 1.38 | — |
| | t = 6 mo | 3.9 | 88.0 | 6.5 | 1.44 | — |

Based on the purity analysis presented in Table 2, it is apparent that 20 mg/mL dalbavancin formulations in acetate buffer stored at 5±3° C. (5° C.) showed a high level of stability and a high resistance to degradation when stored for at least six months, with or without metal ions. In particular, each of the formulations had less than 1.5% (chromatographic area) of the problematic MAG impurity and less than 2% total impurities as provided for in Table 1. The component distribution of B₀ compared to the other components was high, with B₀ of at least 88% in all cases. In addition, longer storage periods generally did not cause the appearance of any additional degradation products over time.

Table 3 below sets forth the stability results of dalbavancin formulations in acetate buffer prepared as described above. The formulations were stored at 25±2° C. (room temperature), 60±5% RH for 1 month, 2 months, 3 months and 6 months.

TABLE 3
Stability of 20 mg/mL dalbavancin formulations in acetate
buffer, pH 4.5 following extended storage at 25° C., 60% RH

| | | | 1 | 2 | 3 | 6 |
|---|---|---|---|---|---|---|
| | Testing | T₀ | month | months | months | months |
| buffer only | Appearance | cl, co | cl, co | cl, co | cl, co | cl, co |
| | pH | 4.46 | 4.52 | 4.54 | 4.58 | 4.39 |
| | Assay % | 102.8 | 99.5 | 100.0 | 94.2 | 93.7 |
| | Chromatographic purity (%) | 97.97 | 97.26 | 96.61 | 95.73 | 92.79 |
| +2 mM Ca²⁺ | Appearance | cl, co | cl, co | cl, co | cl, co | cl, co |
| | pH | 4.41 | 4.51 | 4.51 | 4.54 | 4.50 |
| | Assay % | 101.1 | 99.8 | 99.4 | 94.8 | N/A |
| | Chromatographic purity (%) | 97.94 | 97.75 | 96.67 | 95.70 | 92.79 |
| +10 mM Ca²⁺ | Appearance | cl, co | cl, co | cl, co | cl, co | cl, co |
| | pH | 4.40 | 4.50 | 4.50 | 4.50 | 4.48 |
| | Assay % | 104.2 | 99.3 | 100.0 | 94.0 | 93.1 |
| | Chromatographic purity (%) | 97.95 | 97.16 | 96.41 | 95.55 | 92.65 |
| +2 mM Mg²⁺ | Appearance | cl, co | cl, co | cl, co | cl, co | cl, co |
| | pH | 4.45 | 4.48 | 4.52 | 4.51 | 4.51 |
| | Assay % | 101.8 | 101.3 | 99.2 | 95.2 | 93.2 |
| | Chromatographic purity (%) | 97.96 | 97.25 | 96.61 | 95.67 | 92.69 |
| +2 mM Zn²⁺ | Appearance | cl, co | cl, co | cl, co | cl, co | cl, co |
| | pH | 4.41 | 4.50 | 4.50 | 4.51 | 4.50 |
| | Assay % | 101.7 | 102.5 | 99.8 | 94.3 | 93.4 |
| | Chromatographic purity (%) | 97.94 | 97.25 | 96.62 | 95.68 | 92.73 |

Solution appearance (cl = clear; co = colorless; y = slight yellow color; p = slight pink color; sh = slightly hazy; h = hazy; ppt = precipitated)

As seen from Table 3, dalbavancin formulations in acetate buffer were all stable by appearance, pH and by both chromatographic tests for at least six months when stored at 25° C., 60% RH. All of the tested samples demonstrated less than 3% dalbavancin degradation (greater than 97% sample purity by the chromatographic purity testing) following 1 month of storage and less than 8% dalbavancin degradation following 6 months of room temperature storage. The samples were similar in overall stability regardless of the choice of ion or ion concentration of the test sample. The analysis of component distribution and degradation products that were identified from the HPLC assay experiments for 20 mg/mL dalbavancin formulations in 10 mM acetate buffer, pH 4.5 following extended storage at 25° C., 60% RH are set forth in Table 4.

TABLE 4
Chromatographic purity analysis of stability/purity of 20 mg/mL
dalbavancin formulations in acetate buffer, pH 4.5 following extended
storage at 25° C., 60% RH for t = 0, 1, 2, 3 and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| Buffer only | t = 0 | 4.1 | 92.5 | 6.1 | 1.19 | 0.84 |
| | t = 1 mo | 4.2 | 89.9 | 5.5 | 2.21 | 0.53 |
| | t = 2 mo | 4.0 | 86.9 | 5.8 | 3.39 | — |
| | t = 3 mo | 3.7 | 86.1 | 5.9 | 4.27 | — |
| | t = 6 mo | 3.7 | 83.2 | 5.9 | 7.23 | — |
| +2 mM Ca²⁺ | t = 0 | 4.1 | 90.9 | 6.0 | 1.20 | 0.86 |
| | t = 1 mo | 4.0 | 90.1 | 5.7 | 2.28 | 0.53 |
| | t = 2 mo | 3.9 | 86.8 | 5.9 | 3.33 | — |
| | t = 3 mo | 3.8 | 86.1 | 5.9 | 4.30 | — |
| | t = 6 mo | 3.7 | 83.3 | 5.9 | 7.21 | — |
| +10 mM Ca²⁺ | t = 0 | 4.2 | 93.8 | 6.2 | 1.21 | 0.84 |
| | t = 1 mo | 3.9 | 89.5 | 5.9 | 2.28 | 0.56 |
| | t = 2 mo | 4.0 | 86.7 | 5.8 | 3.59 | — |
| | t = 3 mo | 3.8 | 85.9 | 5.9 | 4.45 | — |
| | t = 6 mo | 3.7 | 83.1 | 5.9 | 7.35 | — |
| +2 mM Mg²⁺ | t = 0 | 4.1 | 91.7 | 6.0 | 1.20 | 0.84 |
| | t = 1 mo | 3.9 | 91.4 | 6.0 | 2.21 | 054 |
| | t = 2 mo | 3.9 | 86.9 | 5.8 | 3.39 | — |
| | t = 3 mo | 3.8 | 86.0 | 5.9 | 4.33 | — |
| | t = 6 mo | 3.7 | 83.2 | 5.9 | 7.31 | — |
| +2 mM Zn²⁺ | t = 0 | 4.1 | 91.5 | 6.1 | 1.21 | 0.85 |
| | t = 1 mo | 4.2 | 92.2 | 6.2 | 2.22 | 0.53 |
| | t = 2 mo | 4.0 | 89.3 | 5.4 | 3.38 | — |
| | t = 3 mo | 3.8 | 86.0 | 5.9 | 4.32 | — |
| | t = 6 mo | 3.6 | 83.2 | 5.9 | 7.27 | — |

In comparing the degradation data presented in Table 4, it is apparent that 20 mg/mL dalbavancin formulations in acetate buffer with or without metal ions stored at 25° C., 60% RH for at least 6 months showed a high level of stability and a high resistance to degradation. In particular, at 1 month, each of the formulations had less than 2.5% (chromatographic area) of MAG and less than 3% total impurities. At 6 months, more MAG was present (about 7%) but was still well within acceptable levels. The component distribution of B₀ compared to the other factors was high, with B₀ over 89% in all cases following 1 month of storage. Following 6 months of room temperature storage, the B₀ component was still above 83% and remained the primary dalbavancin component present. Notably, following extended storage times, the A₀+A₁ component was largely the same (about 3.6 to 4.0%) and the B₁+B₂ component increased as the B₀ component decreased. The formulations that included Zn²⁺ ions provided for a higher B₀ proportion as compared to either buffer alone or the other metal ions tested at both the 1-month and 2-month time points.

Table 5 below sets forth the stability results of dalbavancin formulations in acetate buffer prepared as described above. The formulations were stored at 40±2° C. (room temperature), 75±5% RH for 1 month, 2 months, 3 months and 6 months. Table 6 present stability/purity data for t=0 and t=1 month for high (40° C.) temperature storage.

TABLE 5
Stability of 20 mg/mL dalbavancin formulations in acetate
buffer, pH 4.5 following extended storage at 40° C., 75% RH

| | | | 1 | 2 | 3 | 6 |
|---|---|---|---|---|---|---|
| form | Testing | T₀ | month | months | months | months |
| buffer only | Appearance | cl, co | cl, y | cl, y | cl, y | cl, dy |
| | pH | 4.46 | 4.58 | 4.54 | 4.62 | 4.49 |
| | Assay % | 102.8 | 88.6 | 75.9 | 63.8 | 48.4 |
| | Chromatographic purity (%) | 97.97 | 88.50 | 79.31 | 71.3 | 53.77 |
| +2 mM Ca²⁺ | Appearance | cl, co | cl, y | cl, y | cl, y | cl, dy |
| | pH | 4.41 | 4.54 | 4.53 | 4.58 | 4.50 |
| | Assay % | 101.1 | 86.6 | 77.5 | 63.2 | 49.0 |
| | Chromatographic purity (%) | 97.94 | 98.67 | 79.21 | 71.34 | 58.02 |
| +10 mM Ca²⁺ | Appearance | cl, co | cl, y | cl, y | cl, y | cl, dy |
| | pH | 4.40 | 4.49 | 4.55 | 4.55 | 4.48 |
| | Assay % | 104.2 | 88.4 | 76.7 | 63.5 | 50.7 |
| | Chromatographic purity (%) | 97.95 | 87.84 | 79.29 | 71.65 | 58.89 |
| +2 mM Mg²⁺ | Appearance | cl, co | cl, y | cl, y | cl, y | cl, dy |
| | pH | 4.45 | 4.48 | 4.55 | 4.60 | 4.54 |
| | Assay % | 101.8 | 88.3 | 745.4 | 64.4 | 63.2 |
| | Chromatographic purity (%) | 97.96 | 88.45 | 79.26 | 71.21 | 57.42 |
| +2 mM Zn²⁺ | Appearance | cl, co | cl, y | cl, y | cl, y | cl, dy |
| | pH | 4.41 | 4.49 | 4.55 | 4.57 | 4.51 |
| | Assay % | 101.7 | 89.1 | 76.8 | 63.2 | 48.7 |
| | Chromatographic purity (%) | 97.94 | 88.34 | 79.05 | 70.26 | 56.84 |

Solution appearance (cl = clear; co = colorless; y = slight yellow color; p = slight pink color; sh = slightly hazy; h = hazy; ppt = precipitated)

TABLE 6
Chromatographic purity analysis of stability/purity of 20
mg/mL dalbavancin formulations in acetate buffer, pH 4.5
following extended storage at 40° C., 75% RH for t = 1 month

| | | dalbavancin component | | | | |
|---|---|---|---|---|---|---|
| | | distribution (%) | | | % degradation products | |
| | | A₀ + A₁ | B₀ | B₁ + B₂ | MAG | other |
| Buffer only | t = 0 | 4.1 | 92.5 | 6.1 | 1.19 | 0.84 |
| | t = 1 mo | 3.5 | 79.8 | 5.3 | 10.54 | 0.96 |
| +2 mM Ca²⁺ | t = 0 | 4.1 | 90.9 | 6.0 | 1.20 | 0.86 |
| | t = 1 mo | 3.5 | 79.0 | 5.1 | 10.77 | 0.66 |
| +10 mM Ca²⁺ | t = 0 | 4.2 | 93.8 | 6.2 | 1.21 | 0.84 |
| | t = 1 mo | 3.9 | 79.6 | 5.1 | 11.22 | 0.94 |
| +2 mM Mg²⁺ | t = 0 | 4.1 | 91.7 | 6.0 | 1.20 | 0.84 |
| | t = 1 mo | 3.5 | 80.4 | 5.3 | 10.62 | 0.92 |
| +2 mM Zn²⁺ | t = 0 | 4.1 | 91.5 | 6.1 | 1.21 | 0.85 |
| | t = 1 mo | 3.6 | 80.2 | 5.3 | 10.81 | 0.75 |

Although the formulations stored at 40° C., 75% RH conditions met the visual inspection requirements of being a clear solution with no noticeable precipitation of solid material, the formulations after being stored for 1 month, all had a hint of yellow to the transparency. As seen in Table 5, all of the formulations containing metal ions met the pH stability requirement, in this case all were within ±0.1 pH unit, even following 6 months of storage. But all of the formulations failed both chromatography tests following 1 month of storage at 40° C., 75% RH and extended storage times showed even further degradation of dalbavancin. The analysis of component distribution and degradation products for the 40° C., 75% RH storage conditions after 1 month are set forth in Table 6. In all cases, the MAG was observed at levels higher than 10% (chromatographic area). In addition, small levels of DB-R6 (RT approximately 48 minutes, RRT=1.26), which had not been observed following storage at the other conditions, were present in some of the formulations (included in “other” category in Table 6).

To understand the influence of dalbavancin concentration on the stability of formulations stored in acetate buffer with and without metal ions, formulations containing 1 mg/mL and 5 mg/mL dalbavancin were prepared as provided above. Table 7 provides the overall stability of 1 mg/mL and 5 mg/mL dalbavancin in 10 mM acetate buffer with and without metal ions.

TABLE 7
Stability of 1 and 5 mg/mL dalbavancin formulations in 10 mM acetate buffer,
pH 4.5 following extended storage at 5 ± 3° C.

| | | | 1 | 2 | 3 | 6 |
|---|---|---|---|---|---|---|
| | Testing | T₀ | month | months | months | months |
| buffer only, | Appearance | cl, co | cl, co | cl, co | cl, co | cl, co |
| 1 mg/mL | pH | 4.58 | 4.60 | 4.56 | 4.57 | 4.9 |
| dalbavancin | Assay % | 96.2 | 99.5 | 101.8 | 97.1 | 100.3 |
| | Chromatographic | 97.95 | 97.95 | 98.68 | 98.43 | 98.77 |
| | purity (%) | | | | | |
| 1 mg/mL | Appearance | cl, co | cl, co | cl, co | cl, co | cl, co |
| dalbavancin + | pH | 4.47 | 4.54 | 4.46 | 4.49 | 4.50 |
| 10 mM Ca²⁺ | Assay % | 96.3 | 100.8 | 101.4 | 97.0 | 99.6 |
| | Chromatographic | 97.92 | 98.16 | 98.6 | 98.63 | 98.58 |
| | purity (%) | | | | | |
| 5 mg/mL | Appearance | cl, co | cl, co | cl, co | cl, co | cl, y |
| dalbavancin + | pH | 4.44 | 4.46 | 4.41 | 4.43 | 4.43 |
| 10 mM Ca²⁺ | Assay % | 98.1 | 100.2 | 98.4 | 96.9 | 91.1 |
| | Chromatographic | 97.93 | 98.29 | 97.72 | 98.61 | 98.71 |
| | purity (%) | | | | | |
| 1 mg/mL | Appearance | cl, co | cl, co | cl, co | cl, co | cl, co |
| dalbavancin + | pH | 4.49 | 4.55 | 4.48 | 4.49 | 4.52 |
| 10 mM Mg²⁺ | Assay % | 97.5 | 99.4 | 101.2 | 97.3 | 100.0 |
| | Chromatographic | 97.92 | 98.16 | 98.69 | 98.64 | 98.58 |
| | purity (%) | | | | | |
| 1 mg/mL | Appearance | cl, co | cl, co | cl, co | cl, co | cl, co |
| dalbavancin + | pH | 4.48 | 4.58 | 4.50 | 4.51 | 4.55 |
| 5 mM Zn²⁺ | Assay % | 92.6 | 100.8 | 100.9 | 97.3 | 100.0 |
| | Chromatographic | 97.92 | 98.18 | 98.76 | 98.64 | 98.61 |
| | purity (%) | | | | | |

Solution appearance (cl = clear; co = colorless; y = slight yellow color; p = slight pink color; sh = slightly hazy; h = hazy; ppt = precipitated)

As seen from Table 7, regardless of concentration, dalbavancin formulations in acetate buffer were all stable by appearance, pH and by both chromatographic tests for at least six months when stored at 5±3° C. All of the tested samples demonstrated less than 2% sample degradation (greater than 98% sample purity by the chromatographic purity testing) following 6 months of storage. There was no noticeable difference in stability between 1 mg/mL, 5 mg/mL and 20 mg/mL (Table 1 above) following refrigerated storage for 6 months.

Table 8 below sets forth the analysis of component distribution and degradation products that were identified from the HPLC assay experiments for 1 and 5 mg/mL dalbavancin formulations in 10 mM acetate buffer, pH 4.5 at t=0 and following extended storage at 5±3° C. for 1 month and for 6 months.

TABLE 8
Chromatographic purity analysis of stability/purity of 1 mg/mL
and 5 mg/mL dalbavancin formulations in acetate buffer, pH 4.5
following extended storage at 5° C. for t = 0, 1 and 6 months

| | | dalbavancin component | | | | |
|---|---|---|---|---|---|---|
| | | distribution (%) | | | % degradation products | |
| | | A₀ + A₁ | B₀ | B₁ + B₂ | MAG | other |
| buffer only, | t = 0 | 3.9 | 86.7 | 5.7 | 1.21 | 0.84 |
| 1 mg/mL | t = 1 mo | 4.0 | 89.5 | 6.0 | 1.23 | 0.56 |
| dalbavancin | t = 6 mo | 3.9 | 88.4 | 6.3 | 1.37 | — |
| 1 mg/mL | t = 0 | 3.9 | 86.8 | 5.6 | 1.21 | 0.87 |
| dalbavancin + | t = 1 mo | 4.0 | 90.5 | 6.2 | 1.28 | 0.56 |
| 10 mM Ca²⁺ | t = 6 mo | 3.9 | 88.4 | 6.3 | 1.42 | — |
| 5 mg/mL | t = 0 | 4.0 | 88.4 | 5.8 | 1.18 | 0.89 |
| dalbavancin + | t = 1 mo | 4.0 | 90.2 | 5.9 | 1.28 | 0.43 |
| 10 mM Ca²⁺ | t = 6 mo | 3.6 | 85.8 | 9.2 | 1.39 | — |
| 1 mg/mL | t = 0 | 4.0 | 87.8 | 5.7 | 1.21 | 0.87 |
| dalbavancin + | t = 1 mo | 3.9 | 89.8 | 5.7 | 1.28 | 0.43 |
| 10 mM Mg²⁺ | t = 6 mo | 3.9 | 88.5 | 6.2 | 1.42 | — |
| 1 mg/mL | t = 0 | 3.8 | 83.5 | 5.4 | 1.19 | 0.89 |
| dalbavancin + | t = 1 mo | 3.8 | 90.7 | 6.2 | 1.28 | 0.54 |
| 5 mM Zn²⁺ | t = 6 mo | 3.9 | 88.4 | 6.4 | 1.39 | — |

As compared to Table 7, less concentrated dalbavancin formulations (1 m/mL and 5 mg/mL) in acetate buffer following extended storage at refrigerated temperatures demonstrated similar levels of stability and resistance to degradation. Regardless of concentration of dalbavancin, all formulations demonstrated marked stability (greater than about 88% B₀, greater than 92% total dalbavancin components) and limited degradation (less than 1.5% of MAG) even following storage of at least 6 months. Other than the formulation containing 5 mg/mL dalbavancin and 10 mM Ca²⁺ ions, all of the formulations after 6 months of extended storage contained at least 88% of the B₀ component relative to the total dalbavancin present in the formulation. The 5 mg/mL with 10 mM Ca²⁺ had a slightly lower amount of the B₀ component present, 85.8 at 6 months, however still within the acceptable range.

Accelerated storage conditions for the lower concentration dalbavancin formulations were also tested. Tables 9 and 10 show the experimental data for 1 mg/mL and 5 mg/mL formulations stored at 25±2° C., 60±5% RH, while Tables 11 and 12 provide data for formulations stored at 40±2° C., 75% RH.

TABLE 9
Stability of 1 and 5 mg/mL dalbavancin formulations in 10 mM acetate
buffer, pH 4.5 following extended storage at 25° C., 60% RH

| | | | 1 | 2 | 3 | 6 |
|---|---|---|---|---|---|---|
| | Testing | T₀ | month | months | months | months |
| buffer only, | Appearance | cl, co | cl, co | cl, co | cl, co | cl, co |
| 1 mg/mL | pH | 4.58 | 4.63 | 4.61 | 4.58 | 4.64 |
| dalbavancin | Assay % | 96.2 | 98.6 | 99.5 | 93.8 | 95.3 |
| | Chromatographic | 97.95 | 97.66 | 97.59 | 96.94 | 95.22 |
| | purity (%) | | | | | |
| 1 mg/mL | Appearance | cl, co | cl, co | cl, co | cl, co | cl, co |
| dalbavancin + | pH | 4.47 | 4.54 | 4.45 | 4.49 | 4.46 |
| 10 mM Ca²⁺ | Assay % | 96.3 | 99.0 | 98.9 | 91.5 | 94.2 |
| | Chromatographic | 97.92 | 97.41 | 97.08 | 96.12 | 93.50 |
| | purity (%) | | | | | |
| 5 mg/mL | Appearance | cl, co | cl, co | cl, co | cl, co | cl, co |
| dalbavancin + | pH | 4.44 | 4.49 | 4.44 | 4.44 | 4.44 |
| 10 mM Ca²⁺ | Assay % | 98.1 | 96.7 | 97.2 | 93.0 | 84.3 |
| | Chromatographic | 97.93 | 97.36 | 96.79 | 95.66 | 93.20 |
| | purity (%) | | | | | |
| 1 mg/mL | Appearance | cl, co | cl, co | cl, co | cl, co | cl, co |
| dalbavancin + | pH | 4.49 | 4.55 | 4.55 | 4.52 | 4.51 |
| 10 mM Mg²⁺ | Assay % | 97.5 | 98.1 | 98.7 | 93.0 | 94.3 |
| | Chromatographic | 97.92 | 97.38 | 96.90 | 96.12 | 93.51 |
| | purity (%) | | | | | |
| 1 mg/mL | Appearance | cl, co | cl, co | cl, co | cl, co | cl, co |
| dalbavancin + | pH | 4.48 | 4.59 | 4.54 | 4.53 | 4.56 |
| 5 mM Zn²⁺ | Assay % | 92.6 | 100.1 | 99.4 | 93.2 | 94.4 |
| | Chromatographic | 97.92 | 97.49 | 97.10 | 96.46 | 95.25 |
| | purity (%) | | | | | |

Solution appearance (cl = clear; co = colorless; y = slight yellow color; p = slight pink color; sh = slightly hazy; h = hazy; ppt = precipitated)

TABLE 10
Chromatographic purity analysis of stability/purity of 1 mg/mL and
5 mg/mL dalbavancin formulations in acetate buffer, pH 4.5 following
extended storage at 25° C., 60% RH for 0, 1 and 6 months

| | | dalbavancin component | | | | |
|---|---|---|---|---|---|---|
| | | distribution (%) | | | % degradation products | |
| | | A₀ + A₁ | B₀ | B₁ + B₂ | MAG | other |
| buffer only, | t = 0 | 3.9 | 86.7 | 5.7 | 1.21 | 0.84 |
| 1 mg/mL | t = 1 mo | 4.0 | 88.8 | 6.0 | 1.79 | 0.55 |
| dalbavancin | t = 6 mo | 3.7 | 85.1 | 6.0 | 4.66 | 0.22 |
| 1 mg/mL | t = 0 | 3.9 | 86.8 | 5.6 | 1.21 | 0.87 |
| dalbavancin + | t = 1 mo | 4.0 | 89.1 | 5.8 | 2.08 | 0.51 |
| 10 mM Ca²⁺ | t = 6 mo | 3.7 | 83.6 | 5.9 | 6.17 | 0.33 |
| 5 mg/mL | t = 0 | 4.0 | 88.4 | 5.8 | 1.18 | 0.89 |
| dalbavancin + | t = 1 mo | 4.0 | 87.0 | 5.7 | 2.21 | 0.43 |
| 10 mM Ca²⁺ | t = 6 mo | 3.5 | 81.2 | 8.5 | 6.8 | — |
| 1 mg/mL | t = 0 | 4.0 | 87.8 | 5.7 | 1.21 | 0.87 |
| dalbavancin + | t = 1 mo | 4.2 | 87.9 | 6.0 | 2.07 | 0.55 |
| 10 mM Mg²⁺ | t = 6 mo | 3.7 | 83.8 | 5.9 | 6.16 | 0.33 |
| 1 mg/mL | t = 0 | 3.8 | 83.5 | 5.4 | 1.19 | 0.89 |
| dalbavancin + | t = 1 mo | 3.8 | 90.7 | 6.2 | 1.99 | 0.52 |
| 5 mM Zn²⁺ | t = 6 mo | 3.7 | 84.3 | 6.0 | 5.54 | 0.21 |

TABLE 11
Stability of 1 and 5 mg/mL dalbavancin formulations in 10 mM acetate
buffer, pH 4.5 following extended storage at 40° C., 75% RH

| | | | 1 | 2 | 3 | 6 |
|---|---|---|---|---|---|---|
| | Testing | T₀ | month | months | months | months |
| buffer only, | Appearance | cl, co | cl, co | cl, co | cl, co | cl, co |
| 1 mg/mL | pH | 4.58 | 4.64 | 4.62 | 4.60 | 4.63 |
| dalbavancin | Assay % | 96.2 | 91.5 | 83.1 | 70.5 | 56.3 |
| | Chromatographic | 97.95 | 97.66 | 86.3 | 78.87 | 65.76 |
| | purity (%) | | | | | |
| 1 mg/mL | Appearance | cl, co | cl, co | cl, co | cl, co | cl, co |
| dalbavancin + | pH | 4.47 | 4.54 | 4.53 | 4.51 | 4.46 |
| 10 mM Ca²⁺ | Assay % | 96.3 | 90.3 | 79.9 | 67.6 | 50.6 |
| | Chromatographic | 97.92 | 97.41 | 82.62 | 75.33 | 59.57 |
| | purity (%) | | | | | |
| 5 mg/mL | Appearance | cl, co | cl, co | cl, co | cl, co | cl, y |
| dalbavancin + | pH | 4.44 | 4.51 | 4.49 | 4.48 | 4.43 |
| 10 mM Ca²⁺ | Assay % | 98.1 | 86.9 | 74.4 | 62.2 | 43.6 |
| | Chromatographic | 97.93 | 97.36 | 79.38 | 71.13 | 56.92 |
| | purity (%) | | | | | |
| 1 mg/mL | Appearance | cl, co | cl, co | cl, co | cl, co | cl, co |
| dalbavancin + | pH | 4.49 | 4.56 | 4.54 | 4.52 | 4.50 |
| 10 mM Mg²⁺ | Assay % | 97.5 | 89.5 | 78.9 | 67.4 | 49.9 |
| | Chromatographic | 97.92 | 97.38 | 82.23 | 74.43 | 58.9 |
| | purity (%) | | | | | |
| 1 mg/mL | Appearance | cl, co | cl, co | cl, co | cl, co | cl, co |
| dalbavancin + | pH | 4.48 | 4.62 | 4.57 | 4.53 | 4.50 |
| 5 mM Zn²⁺ | Assay % | 92.6 | 90.3 | 81.2 | 68.6 | 52.8 |
| | Chromatographic | 97.92 | 97.49 | 83.77 | 76.66 | 61.19 |
| | purity (%) | | | | | |

Solution appearance (cl = clear; co = colorless; y = slight yellow color; p = slight pink color; sh = slightly hazy; h = hazy; ppt = precipitated)

TABLE 12
Chromatographic purity analysis of stability/purity of 1 mg/mL and
5 mg/mL dalbavancin formulations in acetate buffer, pH 4.5 following
extended storage at 40° C., 75% RH for t = 0 and t = 1 month

| | | dalbavancin component | | | | |
|---|---|---|---|---|---|---|
| | | distribution (%) | | | % degradation products | |
| | | A₀ + A₁ | B₀ | B₁ + B₂ | MAG | other |
| buffer only, | t = 0 | 3.9 | 86.7 | 5.7 | 1.21 | 0.84 |
| 1 mg/mL | t = 1 mo | 3.6 | 82.6 | 5.4 | 7.07 | — |
| dalbavancin | | | | | | |
| 1 mg/mL | t = 0 | 3.9 | 86.8 | 5.6 | 1.21 | 0.87 |
| dalbavancin + | t = 1 mo | 3.5 | 81.7 | 5.1 | 9.07 | — |
| 10 mM Ca²⁺ | | | | | | |
| 5 mg/mL | t = 0 | 4.0 | 88.4 | 5.8 | 1.18 | 0.89 |
| dalbavancin + | t = 1 mo | 3.4 | 78.5 | 5.0 | 10.72 | — |
| 10 mM Ca²⁺ | | | | | | |
| 1 mg/mL | t = 0 | 4.0 | 87.8 | 5.7 | 1.21 | 0.87 |
| dalbavancin + | t = 1 mo | 3.6 | 80.8 | 5.2 | 9.28 | — |
| 10 mM Mg²⁺ | | | | | | |
| 1 mg/mL | t = 0 | 3.8 | 83.5 | 5.4 | 1.19 | 0.89 |
| dalbavancin + | t = 1 mo | 3.7 | 81.5 | 5.2 | 8.42 | — |
| 5 mM Zn²⁺ | | | | | | |

While all of the lower concentration formulations were stable at 6 months following room temperature storage, the none of the formulations stored at 40° C., 75% RH were stable at even the 1-month time point. All of the formulations stored at 40° C., 75% RH demonstrated unacceptably high levels of degradation products, including MAG, and unacceptably low levels of dalbavancin, particularly the B₀ component. A comparison of the three different concentrations monitored did not reflect a significant dependence on dalbavancin concentration in terms of its long-term stability at high temperature and humidity, and like the higher concentration formulations, show that temperature and humidity conditions are the primary driver in long-term stability of formulated dalbavancin solutions.

### Example 2—Influence of Buffer on the Stability of Dalbavancin Formulations

In order to understand the importance of the buffer system on the stability of dalbavancin in long term storage conditions, dalbavancin formulations in citrate buffer and phosphate buffer were studied. Formulations were made and studies substantially as described above using 5 mM citrate buffer (pH 4.5) or 10 mM phosphate buffer (pH 7.0).

TABLE 13
Stability of 1 mg/mL dalbavancin formulations
in 5 mM citrate buffer, pH 4.5 following extended
storage at 5° C. for t = 1 mo

| | | | | t = 1 mo | | | t = 1 mo | | |
|---|---|---|---|---|---|---|---|---|---|
| | t = 0 | | | 5° C. | | | 55° C. | | |
| | A | B | C | A | B | C | A | B | C |
| 5 mM citrate buffer | 84 | 94 | cl | 85 | 95 | cl | <5 | <5 | y, ppt |
| +2 mM CaCl₂ | 84 | 94 | cl | 85 | 94 | ppt | <5 | <5 | y, ppt |
| +5 mM CaCl₂ | 84 | 94 | cl | 84 | 94 | ppt | <5 | <5 | y, ppt |
| +10 mM CaCl₂ | 84 | 94 | cl | 85 | 95 | cl | <5 | <5 | y, ppt |
| +50 mM CaCl₂ | 84 | 94 | cl | 85 | 95 | ppt | 7 | 8 | y, ppt |
| +2 mM MgCl₂ | 84 | 94 | cl | 86 | 96 | ppt | <5 | <5 | y, ppt |
| +5 mM MgCl₂ | 84 | 94 | cl | 85 | 95 | cl | <5 | <5 | y, ppt |
| +10 mM MgCl₂ | 84 | 94 | cl | 84 | 94 | ppt | <5 | <5 | y, ppt |
| +50 mM MgCl₂ | 84 | 94 | cl | 84 | 93 | ppt | <5 | <5 | y, ppt |
| +2 mM ZnCl₂ | 84 | 94 | cl | 86 | 95 | ppt | <5 | <5 | y, ppt |
| +5 mM ZnCl₂ | 84 | 94 | cl | 85 | 95 | cl | <5 | <5 | y, ppt |
| +10 mM ZnCl₂ | 84 | 94 | cl | 84 | 94 | ppt | <5 | <5 | y, ppt |
| +50 mM ZnCl₂ | 84 | 94 | cl | 84 | 94 | ppt | 19 | 21 | y, ppt |

A = % B₀ present in formulation
B = % Total components (A₀, A₁, B₀, B₁, B₂)
C = Solution appearance (cl = clear; co = colorless; y = slight yellow color; p = slight pink color; sh = slightly hazy; h = hazy; ppt = precipitated)

As can be seen from Table 13, the dalbavancin formulations in acetate buffer demonstrated significantly improved stability as compared to formulations in 5 mM citrate buffer. In nearly all of the conditions studied, regardless of the type of metal ion added or the concentration of the metal ion, the citrate-buffered formulations nearly all precipitated after 1 month of storage even in refrigerated conditions, although all formulations remained within acceptable limits for total component (>90% in all cases) and contained similar levels of B₀ as compared with acetate buffer. Following accelerated storage conditions at 55° C. for 1 month, all of the citrate-buffered dalbavancin formulations had a noticeable light-yellow hue and showed evidence of precipitation. Only one of the citrate-buffered formulations (containing 50 mM ZnCl₂) maintained more than 10% of the initial dalbavancin, while most contained less than 5% of the initial amount.

An otherwise identical study employing 10 mM citrate-buffered dalbavancin formulations was conducted (data not shown). In all cases, the B₀ and total components levels were comparable to the 5 mM citrate buffer formulations, but in every case, the 10 mM formulation showed observable levels of precipitation following 1 month of 5° C.

Finally, attempts to make a phosphate-buffered dalbavancin formulation (20 mg/mL dalbavancin, 10 mM phosphate buffer, pH 7.0) failed. Even at t=0, the % B₀ and total components levels were unacceptably low (64% and 71%, respectively) and the formulation had observable precipitation.

### Example 3—Influence of Cyclodextrin on the Stability of Dalbavancin Formulations in Acetate Buffer

In order to further improve the stability of dalbavancin formulations for long-term storage, the influence of cyclodextrin was studied. Dalbavancin formulations in acetate buffer were made substantially as detailed above. To each formulation was added 2-hydroxypropyl-β-cyclodextrin (HPβCD) in a concentration of 55 mM or 110 mM. The formulations were tested according to the extended storage conditions described above.

TABLE 14
Stability of 20 mg/mL dalbavancin formulations in 10 mM acetate buffer,
pH 4.5 including HPβCD, following extended storage at 5° C.

| | | | 1 | 2 | 3 | 6 |
|---|---|---|---|---|---|---|
| | Testing | T₀ | month | months | months | months |
| +55 mM CD | Appearance | cl, co | cl, co | cl, co | cl, co | cl, co |
| | pH | 4.62 | 4.67 | 4.61 | 4.64 | 4.69 |
| | Assay % | 103.0 | 98.7 | 100.1 | 96.4 | 100.0 |
| | Chromatographic | 97.99 | 98.23 | 98.72 | 98.68 | 98.65 |
| | purity (%) | | | | | |
| +110 mM CD | Appearance | cl, co | cl, co | cl, co | cl, co | cl, co |
| | pH | 4.62 | 4.65 | 4.66 | 4.70 | 4.69 |
| | Assay % | 98.8 | 99.0 | 101.8 | 95.2 | 99.4 |
| | Chromatographic | 97.98 | 98.22 | 98.69 | 98.70 | 98.66 |
| | purity (%) | | | | | |
| +55 mM CD + | Appearance | cl, co | cl, co | cl, co | cl, co | cl, co |
| 10 mM Ca²⁺ | pH | 4.62 | 4.57 | 4.58 | 4.59 | 4.62 |
| | Assay % | 100.6 | 100.6 | 101.2 | 97.8 | 99.5 |
| | Chromatographic | 97.92 | 98.16 | 98.82 | 98.67 | 98.64 |
| | purity (%) | | | | | |
| +110 mM CD + | Appearance | cl, co | cl, co | cl, co | cl, co | cl, co |
| 10 mM Ca²⁺ | pH | 4.59 | 4.59 | 4.60 | 4.62 | 4.61 |
| | Assay % | 101.2 | 99.8 | 99.2 | 95.4 | 99.6 |
| | Chromatographic | 97.98 | 98.19 | 98.64 | 98.64 | 98.64 |
| | purity (%) | | | | | |
| +110 mM CD + | Appearance | cl, co | cl, co | cl, co | cl, co | cl, co |
| 10 mM Mg²⁺ | pH | 4.56 | 4.57 | 4.61 | 4.62 | 4.64 |
| | Assay % | 98.7 | 99.2 | 99.4 | 95.5 | 98.0 |
| | Chromatographic | 97.95 | 98.21 | 98.62 | 98.68 | 98.65 |
| | purity (%) | | | | | |
| +110 mM CD + | Appearance | cl, co | cl, co | cl, co | cl, co | cl, co |
| 10 mM Zn²⁺ | pH | 4.49 | 4.54 | 4.57 | 4.60 | 4.61 |
| | Assay % | 101.1 | 102.5 | 100.5 | 95.3 | 98.4 |
| | Chromatographic | 97.98 | 98.23 | 98.69 | 98.67 | 98.65 |
| | purity (%) | | | | | |

Solution appearance (cl = clear; co = colorless; y = slight yellow color; p = slight pink color; sh = slightly hazy; h = hazy; ppt = precipitated)

As provided in Table 14, dalbavancin formulations including CD provide for stable formulations by all measures following 6 months of storage in aqueous buffer at 5±2° C. The formulations containing CD all demonstrated at least 98% chromatographic purity at the 6-month time point, showing high levels of stability for extended periods of time. Table 15 provides the purity analysis for the CD-containing formulations initially and after 1 month and 6 months of extended storage at 5° C. As can be seen, there is no appreciable difference in either the component distribution or the analysis of degradation products following storage. As provided in Table 15, in all cases, the MAG level following 1 month of storage at 5° C. was less than 1.5% (chromatographic area) and the total percentage of components present (chromatographic area) as greater than 97% for all formulations tested. Following 6 months of storage, MAG levels remained under 2% and the total B₀ component stayed over 8%. All of the formulations were stable by all measures and all of the buffered formulations stabilized dalbavancin in solution for at least 6 months of storage in refrigerated conditions.

TABLE 15
Chromatographic purity analysis of stability/purity of 20 mg/mL
dalbavancin formulations in acetate buffer including HPβCD,
pH 4.5 at t = 0 and t = 1 and 6 months following storage at 5° C.

| | | dalbavancin component | | | | |
|---|---|---|---|---|---|---|
| | | distribution (%) | | | % degradation products | |
| | | A₀ + A₁ | B₀ | B₁ + B₂ | MAG | other |
| +55 mM CD | t = 0 | 4.2 | 92.7 | 6.2 | 1.18 | 0.89 |
| | t = 1 mo | 3.9 | 88.9 | 6.0 | 1.22 | 0.55 |
| | t = 6 mo | 4.0 | 88.2 | 6.5 | 1.35 | — |
| +110 mM CD | t = 0 | 4.0 | 88.9 | 5.9 | 1.19 | 0.83 |
| | t = 1 mo | 3.9 | 89.2 | 5.9 | 1.22 | 0.56 |
| | t = 6 mo | 3.9 | 88.3 | 6.3 | 1.36 | — |
| +55 mM CD + | t = 0 | 4.1 | 90.7 | 5.9 | 1.19 | 0.89 |
| 10 mM Ca²⁺ | t = 1 mo | 4.2 | 90.3 | 6.1 | 1.24 | 0.60 |
| | t = 6 mo | 3.9 | 88.3 | 6.3 | 1.34 | — |
| +110 mM CD + | t = 0 | 4.0 | 91.2 | 6.0 | 1.20 | 0.82 |
| 10 mM Ca²⁺ | t = 1 mo | 4.0 | 91.0 | 6.0 | 1.24 | 0.57 |
| | t = 6 mo | 3.9 | 88.1 | 6.5 | 1.36 | — |
| +110 mM CD + | t = 0 | 4.0 | 88.8 | 5.9 | 1.20 | 0.85 |
| 10 mM Mg²⁺ | t = 1 mo | 4.2 | 89.0 | 6.0 | 1.24 | 0.55 |
| | t = 6 mo | 3.9 | 88.3 | 6.5 | 1.35 | — |
| +110 mM CD + | t = 0 | 4.1 | 91.0 | 6.0 | 1.19 | 0.83 |
| 10 mM Zn²⁺ | t = 1 mo | 4.4 | 91.6 | 6.5 | 1.24 | 0.53 |
| | t = 6 mo | 3.9 | 88.3 | 6.5 | 1.35 | — |

Otherwise identical CD-containing formulations were stored under extended storage conditions of 25° C., 60% RH for 1 mo. Table 16 sets forth the results of the stability testing and Table 17 provides the purity analysis of the formulations following storage. All formulations were stable by all measures following 1 month of storage at accelerated (25° C./60% RH) storage conditions. Every tested formulation was greater than 97% purity (chromatographic area) and no sample had more than 2% by area MAG at 1 month. The formulations stored at room temperature had greater than 94% purity (chromatographic area) and no more than 4.5% MAG at 6 months. The component distribution provides that higher levels of CD and/or the inclusion of metal cations may better stabilize the B₀ component. Notably, the unidentified degradation product having RRT 0.92 is not apparent in the formulations that comprise CD but no metal ions, but is apparent in the formulations comprising CD and metal ions.

TABLE 16
Stability of 20 mg/mL dalbavancin formulations in 10 mM acetate buffer, pH 4.5
including HPβCD, following extended storage at 25° C./60% RH

| | | | 1 | 2 | 3 | 6 |
|---|---|---|---|---|---|---|
| | Testing | T₀ | month | months | months | months |
| +55 mM CD | Appearance | cl, co | cl, co | cl, co | cl, co | cl, co |
| | pH | 4.62 | 4.66 | 4.68 | 4.70 | 4.57 |
| | Assay % | 103.0 | 97.7 | 99.9 | 95.4 | 95.9 |
| | Chromatographic | 97.99 | 97.77 | 97.67 | 97.26 | 95.77 |
| | purity (%) | | | | | |
| +110 mM CD | Appearance | cl, co | cl, co | cl, co | cl, co | cl, co |
| | pH | 4.62 | 4.68 | 4.71 | 4.74 | 4.64 |
| | Assay % | 98.8 | 99.5 | 98.0 | 93.9 | 94.1 |
| | Chromatographic | 97.98 | 97.76 | 96.69 | 97.10 | 95.75 |
| | purity (%) | | | | | |
| +55 mM CD + | Appearance | cl, co | cl, co | cl, co | cl, co | cl, co |
| 10 mM Ca²⁺ | pH | 4.62 | 4.59 | 4.59 | 4.61 | 4.50 |
| | Assay % | 100.6 | 99.9 | 99.3 | 96.3 | 96.7 |
| | Chromatographic | 97.92 | 97.73 | 97.65 | 97.21 | 95.76 |
| | purity (%) | | | | | |
| +110 mM CD + | Appearance | cl, co | cl, co | cl, co | cl, co | cl, co |
| 10 mM Ca²⁺ | pH | 4.59 | 4.64 | 4.61 | 4.64 | 4.69 |
| | Assay % | 101.2 | 100.5 | 98.1 | 94.7 | 95.0 |
| | Chromatographic | 97.98 | 97.74 | 97.56 | 96.97 | 95.77 |
| | purity (%) | | | | | |
| +110 mM CD + | Appearance | cl, co | cl, co | co, ppt | cl, co | cl, co |
| 10 mM Mg²⁺ | pH | 4.56 | 4.62 | 4.62 | 4.75 | 4.95 |
| | Assay % | 98.7 | 99.3 | 98.5 | 95.2 | 95.9 |
| | Chromatographic | 97.95 | 97.74 | 97.62 | 97.22 | 94.85 |
| | purity (%) | | | | | |
| +110 mM CD + | Appearance | cl, co | cl, co | cl, co | cl, co | cl, co |
| 10 mM Zn²⁺ | pH | 4.49 | 4.57 | 4.57 | 4.62 | 4.52 |
| | Assay % | 101.1 | 101.9 | 99.8 | 93.6 | 95.1 |
| | Chromatographic | 97.98 | 97.70 | 97.63 | 97.14 | 95.62 |
| | purity (%) | | | | | |

Solution appearance (cl = clear; co = colorless; y = slight yellow color; p = slight pink color; sh = slightly hazy; h = hazy; ppt = precipitated)

TABLE 17
Chromatographic purity analysis of stability/purity of 20 mg/mL dalbavancin
formulations in acetate buffer including HPβCD, pH 4.5 at t =
0 and t = 1 and 6 months following extended storage at 25° C./60% RH

| | | dalbavancin component | | | | |
|---|---|---|---|---|---|---|
| | | distribution (%) | | | % degradation products | |
| | | A₀ + A₁ | B₀ | B₁ + B₂ | MAG | other |
| +55 mM CD | t = 0 | 4.2 | 92.7 | 6.2 | 1.18 | 0.89 |
| | t = 1 mo | 3.9 | 86.3 | 5.5 | 1.69 | 0.54 |
| | t = 6 mo | 3.8 | 85.9 | 6.2 | 4.14 | 0.09 |
| +110 mM CD | t = 0 | 4.0 | 88.9 | 5.9 | 1.19 | 0.83 |
| | t = 1 mo | 3.9 | 89.8 | 5.9 | 1.71 | 0.56 |
| | t = 6 mo | 3.8 | 85.9 | 6.0 | 4.16 | 0.09 |
| +55 mM CD + | t = 0 | 4.1 | 90.7 | 5.9 | 1.19 | 0.89 |
| 10 mM Ca²⁺ | t = 1 mo | 4.0 | 89.7 | 6.2 | 1.68 | 0.56 |
| | t = 6 mo | 3.8 | 86.0 | 6.0 | 4.24 | — |
| +110 mM CD + | t = 0 | 4.0 | 91.2 | 6.0 | 1.20 | 0.82 |
| 10 mM Ca²⁺ | t = 1 mo | 3.9 | 90.7 | 6.0 | 1.72 | 0.50 |
| | t = 6 mo | 3.8 | 85.9 | 6.0 | 3.95 | 0.28 |
| +110 mM CD + | t = 0 | 4.0 | 88.8 | 5.9 | 1.20 | 0.85 |
| 10 mM Mg²⁺ | t = 1 mo | 4.1 | 89.2 | 5.9 | 1.72 | 0.54 |
| | t = 6 mo | 3.8 | 86.0 | 6.0 | 4.15 | 1.00 |
| +110 mM CD + | t = 0 | 4.1 | 91.0 | 6.0 | 1.19 | 0.83 |
| 10 mM Zn²⁺ | t = 1 mo | 4.1 | 91.4 | 6.3 | 1.74 | 0.56 |
| | t = 6 mo | 3.8 | 85.8 | 6.0 | 4.38 | — |

TABLE 18
Stability of 20 mg/mL dalbavancin formulations in 10 mM acetate buffer, pH 4.5
including HPβCD, following extended storage at 40° C./75% RH

| | | | 1 | 2 | 3 | 6 |
|---|---|---|---|---|---|---|
| | Testing | T₀ | month | months | months | months |
| +55 mM CD | Appearance | cl, co | cl, co | cl, co | cl, co | cl, y |
| | pH | 4.62 | 4.66 | 4.71 | 4.70 | 4.74 |
| | Assay % | 103.0 | 93.5 | 89.6 | 78.8 | 70.6 |
| | Chromatographic | 97.99 | 93.13 | 89.11 | 84.05 | 75.03 |
| | purity (%) | | | | | |
| +110 mM CD | Appearance | cl, co | cl, co | cl, co | cl, co | cl, y |
| | pH | 4.62 | 4.69 | 4.75 | 4.67 | 4.75 |
| | Assay % | 98.8 | 93.7 | 87.8 | 78.5 | 68.6 |
| | Chromatographic | 97.98 | 93.17 | 88.89 | 83.93 | 74.62 |
| | purity (%) | | | | | |
| +55 mM CD + | Appearance | cl, co | cl, co | cl, co | cl, co | cl, y |
| 10 mM Ca²⁺ | pH | 4.62 | 4.60 | 4.59 | 4.63 | 4.62 |
| | Assay % | 100.6 | 95.8 | 90.0 | 80.1 | 71.1 |
| | Chromatographic | 97.92 | 93.35 | 88.88 | 84.15 | 74.89 |
| | purity (%) | | | | | |
| +110 mM CD + | Appearance | cl, co | cl, co | cl, co | cl, co | cl, y |
| 10 mM Ca²⁺ | pH | 4.59 | 4.63 | 4.66 | 4.67 | 4.64 |
| | Assay % | 101.2 | 96.4 | 88.3 | 78.3 | 68.3 |
| | Chromatographic | 97.98 | 92.98 | 88.64 | 83.64 | 74.37 |
| | purity (%) | | | | | |
| +110 mM CD + | Appearance | cl, co | cl, co | cl, co | cl, y | cl, y |
| 10 mM Mg²⁺ | pH | 4.56 | 4.62 | 4.65 | 4.66 | 4.64 |
| | Assay % | 98.7 | 92.8 | 88.7 | 79.5 | 68.9 |
| | Chromatographic | 97.95 | 93.12 | 88.84 | 84.55 | 74.66 |
| | purity (%) | | | | | |
| +110 mM CD + | Appearance | cl, co | cl, co | cl, co | cl, y | cl, y |
| 10 mM Zn²⁺ | pH | 4.49 | 4.59 | 4.62 | 4.64 | 4.60 |
| | Assay % | 101.1 | 95.2 | 86.9 | 77.2 | 67.7 |
| | Chromatographic | 97.98 | 92.4 | 87.54 | 82.24 | 72.18 |
| | purity (%) | | | | | |

Solution appearance (cl = clear; co = colorless; y = slight yellow color; p = slight pink color; sh = slightly hazy; h = hazy; ppt = precipitated)

TABLE 19
Chromatographic purity analysis of stability/purity of 20 mg/mL dalbavancin
formulations in acetate buffer, pH 4.5 including HPβCD at t =
0 and following extended storage at 40° C./75% RH for 1 mo

| | | dalbavancin component | | | | |
|---|---|---|---|---|---|---|
| | | distribution (%) | | | % degradation products | |
| | | A₀ + A₁ | B₀ | B₁ + B₂ | MAG | other |
| +55 mM CD | t = 0 | 4.2 | 92.7 | 6.2 | 1.18 | 0.89 |
| | t = 1 mo | 3.7 | 84.4 | 5.4 | 5.91 | 0.96 |
| +110 mM CD | t = 0 | 4.0 | 88.9 | 5.9 | 1.19 | 0.83 |
| | t = 1 mo | 3.7 | 84.6 | 5.3 | 5.96 | 0.88 |
| +55 mM CD + | t = 0 | 4.1 | 90.7 | 5.9 | 1.19 | 0.89 |
| 10 mM Ca²⁺ | t = 1 mo | 3.9 | 86.1 | 5.7 | 5.97 | 0.68 |
| +110 mM CD + | t = 0 | 4.0 | 91.2 | 6.0 | 1.20 | 0.82 |
| 10 mM Ca²⁺ | t = 1 mo | 4.1 | 86.6 | 5.8 | 6.09 | 0.95 |
| +110 mM CD + | t = 0 | 4.0 | 88.8 | 5.9 | 1.20 | 0.85 |
| 10 mM Mg²⁺ | t = 1 mo | 3.6 | 83.7 | 5.5 | 5.99 | 0.89 |
| +110 mM CD + | t = 0 | 4.1 | 91.0 | 6.0 | 1.19 | 0.83 |
| 10 mM Zn²⁺ | t = 1 mo | 3.8 | 85.8 | 5.7 | 6.63 | 0.98 |

As seen from the above, all formulations comprising 20 mg/mL dalbavancin and CD were stable for at least 1 month when stored at 40° C./75% RH. All formulations remained clear and colorless with no noticeable precipitation by visual inspection. In every case, the pH varied less than 0.1 pH unit over the storage period. The chromatographic purity was higher than 92% in all cases. The analysis of the component distribution showed that B₀ levels remained at least 83% of the total peak area, a decrease of no more than 8% at most following storage. Further, MAG levels, while increased, were still within an acceptable range when the overall composition of the formulation is considered. However, following 2 months of extended storage at 40° C./75% RH, all of the dalbavancin formulations failed the chromatographic purity tests, contained 80% or less of the B₀ component and contained more than 10% of the undesirable MAG impurity (data not shown). It is unexpected to determine that the inclusion of cyclodextrin stabilizes dalbavancin formulations when stored at higher temperatures.

In order to understand the influence of dalbavancin concentration on the stability of the formulation when CD and/or metal ions were added to the formulation, 1 mg/mL and 5 mg/mL dalbavancin formulations were prepared as provided above. To these was added 0.6, 5.5 or 55 mM HPβCD. In some cases, 5% dextrose and/or divalent cations were further added to the formulation. The formulations were stored at either 5° C., 25° C./60% RH or 40° C./75% RH for the specified time periods.

TABLE 20
Stability of 1 mg/mL and 5 mg/mL dalbavancin formulations in 10 mM acetate
buffer, pH 4.5 including HPβCD, following extended storage at 5° C.

| | | | 1 | 2 | 3 | 6 |
|---|---|---|---|---|---|---|
| | Testing | T₀ | month | months | months | months |
| 1 mg/mL | Appearance | cl, co | cl, co | cl, co | cl, co | cl, co |
| dalbavancin + | pH | 4.44 | 4.48 | 4.39 | 4.43 | 4.48 |
| 0.6 mM CD | Assay % | 98.0 | 100.7 | 101.5 | 97.2 | 99.7 |
| | Chromatographic | 97.93 | 98.49 | 98.68 | 98.68 | 98.69 |
| | purity (%) | | | | | |
| 1 mg/mL | Appearance | cl, co | cl, co | cl, co | cl, co | cl, co |
| dalbavancin + | pH | 4.48 | 4.47 | 4.39 | 4.44 | 4.51 |
| 0.6 mM CD + | Assay % | 100.4 | 100.1 | 100.6 | 96.3 | 98.7 |
| 5% dextrose | Chromatographic | 97.94 | 98.54 | 98.60 | 98.68 | 98.69 |
| | purity (%) | | | | | |
| 1 mg/mL | Appearance | cl, co | cl, co | cl, co | cl, co | cl, co |
| dalbavancin + | pH | 4.63 | 4.67 | 4.58 | 4.58 | 4.64 |
| 5.5 mM CD | Assay % | 96.4 | 101.6 | 103.7 | 96.5 | 99.0 |
| | Chromatographic | 97.94 | 98.31 | 98.22 | 98.71 | 98.69 |
| | purity (%) | | | | | |
| 1 mg/mL | Appearance | cl, co | cl, co | cl, co | cl, co | cl, co |
| dalbavancin + | pH | 4.50 | 4.52 | 4.51 | 4.52 | 4.61 |
| 55 mM CD | Assay % | 99.1 | 102.1 | 102.2 | 94.4 | 101.0 |
| | Chromatographic | 98.01 | 98.57 | 98.96 | 98.82 | 98.72 |
| | purity (%) | | | | | |
| 5 mg/mL | Appearance | cl, co | cl, co | cl, co | cl, co | cl, co |
| dalbavancin + | pH | 4.36 | 4.30 | 4.33 | 4.29 | 4.38 |
| 55 mM CD | Assay % | 102.0 | 103.6 | 99.9 | 95.0 | 92.3 |
| | Chromatographic | 97.98 | 98.37 | 98.37 | 98.72 | 98.75 |
| | purity (%) | | | | | |
| 5 mg/mL | Appearance | cl, co | cl, co | cl, co | cl, co | cl, co |
| dalbavancin + | pH | 4.37 | 4.33 | 4.28 | 4.31 | 4.30 |
| 3.0 mM CD + | Assay % | 97.4 | 98.7 | 100.7 | 96.7 | 92.7 |
| 5% dextrose | Chromatographic | 97.95 | 98.44 | 98.44 | 98.68 | 98.76 |
| | purity (%) | | | | | |
| 1 mg/mL | Appearance | cl, co | cl, co | cl, co | cl, co | cl, co |
| dalbavancin + | pH | 4.53 | 4.53 | 4.48 | 4.50 | 4.56 |
| 0.6 mM CD + | Assay % | 94.6 | 99.5 | 100.2 | 95.4 | 98.0 |
| 10 mM Ca²⁺ | Chromatographic | 97.90 | 98.34 | 98.64 | 98.67 | 98.77 |
| | purity (%) | | | | | |
| 1 mg/mL | Appearance | cl, co | cl, co | cl, co | cl, co | cl, co |
| dalbavancin + | pH | 4.56 | 4.54 | 4.48 | 4.50 | 4.56 |
| 0.6 mM CD + | Assay % | 96.7 | 101.2 | 101.8 | 96.6 | 100.2 |
| 10 mM Mg²⁺ | Chromatographic | 97.90 | 98.39 | 98.64 | 98.67 | 98.68 |
| | purity (%) | | | | | |
| 5 mg/mL | Appearance | cl, co | cl, co | cl, co | cl, co | cl, co |
| dalbavancin + | pH | 4.43 | 4.47 | 4.37 | 4.37 | 4.41 |
| 3.0 mM CD + | Assay % | 98.5 | 102.7 | 98.8 | 93.3 | 91.9 |
| 10 mM Mg²⁺ | Chromatographic | 97.95 | 98.31 | 98.46 | 98.68 | 98.75 |
| | purity (%) | | | | | |
| 1 mg/mL | Appearance | cl, co | cl, co | cl, co | cl, co | cl, co |
| dalbavancin + | pH | 4.62 | 4.56 | 4.54 | 4.50 | 4.58 |
| 5.5 mM CD + | Assay % | 99.9 | 101.3 | 101.2 | 97.1 | 100.3 |
| 2.0 mM Zn²⁺ | Chromatographic | 97.92 | 98.29 | 98.41 | 98.68 | 98.68 |
| | purity (%) | | | | | |

Solution appearance (cl = clear; co = colorless; y = slight yellow color; p = slight pink color; sh = slightly hazy; h = hazy; ppt = precipitated)

TABLE 21
Chromatographic purity analysis of stability/purity
of 20 mg/mL dalbavancin formulations in acetate buffer,
pH 4.5 including HPβCD at t = 0 and following
extended storage at 5° C. for 1 month and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| 1 mg/mL | t = 0 | 4.0 | 88.5 | 5.6 | 1.22 | 0.85 |
| dalbavancin + | t = 1 mo | 4.0 | 90.8 | 5.9 | 1.26 | 0.50 |
| 0.6 mM CD | t = 6 mo | 3.9 | 88.6 | 6.2 | 1.31 | — |
| 1 mg/mL | t = 0 | 4.1 | 90.6 | 5.9 | 1.19 | 0.90 |
| dalbavancin + | t = 1 mo | 4.0 | 90.0 | 6.1 | 1.26 | 0.40 |
| 0.6 mM CD + | t = 6 mo | 3.9 | 88.6 | 6.2 | 1.31 | — |
| 5% dextrose | | | | | | |
| 1 mg/mL | t = 0 | 3.9 | 87.0 | 5.6 | 1.18 | 0.88 |
| dalbavancin + | t = 1 mo | 4.0 | 91.1 | 6.4 | 1.22 | 0.47 |
| 5.5 mM CD | t = 6 mo | 3.9 | 88.4 | 6.4 | 1.31 | — |
| 1 mg/mL | t = 0 | 4.0 | 89.4 | 5.8 | 1.16 | 0.83 |
| dalbavancin + | t = 1 mo | 4.3 | 91.6 | 6.2 | 1.20 | 0.23 |
| 55 mM CD | t = 6 mo | 3.9 | 88.5 | 6.4 | 1.28 | — |
| 5 mg/mL | t = 0 | 4.1 | 92.0 | 5.9 | 1.15 | 0.87 |
| dalbavancin + | t = 1 mo | 4.1 | 93.3 | 6.2 | 1.21 | 0.42 |
| 55 mM CD | t = 6 mo | 3.6 | 86.0 | 9.1 | 1.25 | — |
| 5 mg/mL | t = 0 | 4.0 | 87.8 | 5.7 | 1.20 | 0.85 |
| dalbavancin + | t = 1 mo | 3.9 | 89.1 | 5.6 | 1.22 | 0.34 |
| 3.0 mM CD + | t = 6 mo | 3.6 | 85.9 | 9.2 | 1.24 | — |
| 5% dextrose | | | | | | |
| 1 mg/mL | t = 0 | 3.9 | 85.2 | 5.5 | 1.23 | 0.77 |
| dalbavancin + | t = 1 mo | 4.0 | 89.7 | 5.9 | 1.27 | 0.39 |
| 0.6 mM CD + | t = 6 mo | 3.9 | 88.4 | 6.3 | 1.33 | — |
| 10 mM Ca² | | | | | | |
| 1 mg/mL | t = 0 | 4.1 | 87.7 | 5.8 | 1.23 | 0.87 |
| dalbavancin + | t = 1 mo | 4.3 | 90.7 | 6.2 | 1.26 | 0.35 |
| 0.6 mM CD + | t = 6 mo | 3.9 | 88.4 | 6.4 | 1.32 | — |
| 10 mM Mg²⁺ | | | | | | |
| 5 mg/mL | t = 0 | 4.0 | 88.7 | 5.8 | 1.2 | 0.85 |
| dalbavancin + | t = 1 mo | 4.3 | 92.2 | 6.2 | 1.25 | 0.29 |
| 3.0 mM CD + | t = 6 mo | 3.6 | 86.0 | 9.1 | 1.25 | — |
| 10 mM Mg²⁺ | | | | | | |
| 1 mg/mL | t = 0 | 4.0 | 90.1 | 5.9 | 1.20 | 0.88 |
| dalbavancin + | t = 1 mo | 4.2 | 91.1 | 6.1 | 1.24 | 0.50 |
| 5.5 mM CD + | t = 6 mo | 3.9 | 88.4 | 6.3 | 1.32 | — |
| 2.0 mM Zn²⁺ | | | | | | |

As seen in Tables 20 and 21, the lower concentration dalbavancin formulations were all stable following 6 months of storage at 5° C. Overall, the B₀ component levels and the MAG levels were comparable to those observed for the 20 mg/mL dalbavancin formulations under the same storage conditions. All dalbavancin levels and MAG levels were well within acceptable limits.

To understand the effects of storage conditions on the lower concentration dalbavancin formulations, the 1 mg/mL and 5 mg/mL formulations were stored at 25° C./60% RH and 40° C./75% RH for the indicated time periods.

TABLE 22
Stability of 1 mg/mL and 5 mg/mL dalbavancin formulations in 10 mM acetate
buffer, pH 4.5 including HPβCD, following extended storage at 25° C./60% RH

| | | | 1 | 2 | 3 | 6 |
|---|---|---|---|---|---|---|
| | Testing | T₀ | month | months | months | months |
| 1 mg/mL | Appearance | cl, co | cl, co | cl, co | cl, co | cl, co |
| dalbavancin + | pH | 4.44 | 4.47 | 4.40 | 4.42 | 4.50 |
| 0.6 mM CD | Assay % | 98.0 | 100.4 | 101.4 | 96.2 | 98.9 |
| | Chromatographic purity (%) | 97.93 | 98.19 | 98.32 | 98.05 | 97.13 |
| 1 mg/mL | Appearance | cl, co | cl, co | cl, co | cl, co | cl, co |
| dalbavancin + | pH | 4.48 | 4.47 | 4.43 | 4.43 | 4.50 |
| 0.6 mM CD + | Assay % | 100.4 | 100.3 | 100.9 | 95.4 | 98.3 |
| 5% dextrose | Chromatographic purity (%) | 97.94 | 98.12 | 98.22 | 98.05 | 97.33 |
| 1 mg/mL | Appearance | cl, co | cl, co | cl, co | cl, co | cl, co |
| dalbavancin + | pH | 4.63 | 4.58 | 4.58 | 4.60 | 4.62 |
| 5.5 mM CD | Assay % | 96.4 | 100.1 | 100.9 | 95.8 | 98.7 |
| | Chromatographic purity (%) | 97.94 | 98.13 | 98.25 | 97.90 | 96.84 |
| 1 mg/mL | Appearance | cl, co | cl, co | cl, co | cl, co | cl, co |
| dalbavancin + | pH | 4.50 | 4.60 | 4.51 | 4.51 | 4.79 |
| 55 mM CD | Assay % | 99.1 | 101.8 | 101.9 | 96.9 | 99.6 |
| | Chromatographic purity (%) | 98.01 | 97.98 | 98.24 | 97.94 | 96.92 |
| 5 mg/mL | Appearance | cl, co | cl, co | cl, co | cl, co | cl, co |
| dalbavancin + | pH | 4.36 | 4.48 | 4.51 | 4.52 | 4.54 |
| 55 mM CD | Assay % | 102.0 | 100.4 | 98.4 | 93.7 | 90.0 |
| | Chromatographic purity (%) | 97.98 | 97.91 | 97.91 | 97.73 | 96.98 |
| 5 mg/mL | Appearance | cl, co | cl, co | cl, co | cl, co | cl, co |
| dalbavancin + | pH | 4.37 | 4.36 | 4.29 | 4.31 | 4.34 |
| 3.0 mM CD + | Assay % | 97.4 | 102.0 | 100.9 | 96.5 | 91.5 |
| 5% dextrose | Chromatographic purity (%) | 97.95 | 98.34 | 98.19 | 98.06 | 97.47 |
| 1 mg/mL | Appearance | cl, co | cl, co | cl, co | cl, co | cl, co |
| dalbavancin + | pH | 4.53 | 4.54 | 4.46 | 4.49 | 4.46 |
| 0.6 mM CD + | Assay % | 94.6 | 99.6 | 100.5 | 94.7 | 97.1 |
| 10 mM Ca²⁺ | Chromatographic purity (%) | 97.90 | 98.03 | 98.23 | 98.03 | 97.09 |
| 1 mg/mL | Appearance | cl, co | cl, co | cl, co | cl, co | cl, co |
| dalbavancin + | pH | 4.56 | 4.56 | 4.51 | 4.56 | 4.58 |
| 0.6 mM CD + | Assay % | 96.7 | 100.0 | 101.3 | 95.8 | 98.7 |
| 10 mM Mg²⁺ | Chromatographic purity (%) | 97.90 | 97.94 | 98.24 | 98.00 | 97.22 |
| 5 mg/mL | Appearance | cl, co | cl, co | cl, co | cl, co | cl, co |
| dalbavancin + | pH | 4.43 | 4.56 | 4.54 | 4.56 | 4.58 |
| 3.0 mM CD + | Assay % | 98.5 | 100.8 | 98.2 | 94.1 | 88.8 |
| 10 mM Mg²⁺ | Chromatographic purity (%) | 97.95 | 98.19 | 98.19 | 98.05 | 97.47 |
| 1 mg/mL | Appearance | cl, co | cl, co | cl, co | cl, co | cl, co |
| dalbavancin + | pH | 4.62 | 4.57 | 4.57 | 4.53 | 4.55 |
| 5.5 mM CD + | Assay % | 99.9 | 100.7 | 101.3 | 95.9 | 98.7 |
| 2.0 mM Zn²⁺ | Chromatographic purity (%) | 97.92 | 97.89 | 98.15 | 97.88 | 96.80 |

Solution appearance (cl = clear; co = colorless; y = slight yellow color; p = slight pink color; sh = slightly hazy; h = hazy; ppt = precipitated)

TABLE 23
Chromatographic purity analysis of stability/purity of 1
mg/ml and 5 mg/mL dalbavancin formulations in acetate buffer,
pH 4.5 including HPβCD, initially and following extended
storage at 25° C./60% RH for 1 month and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| 1 mg/mL | t = 0 | 4.0 | 88.5 | 5.6 | 1.22 | 0.85 |
| dalbavancin + | t = 1 mo | 4.0 | 90.5 | 5.8 | 1.45 | 0.36 |
| 0.6 mM CD | t = 6 mo | 3.8 | 86.9 | 6.2 | 2.53 | 0.34 |
| 1 mg/mL | t = 0 | 4.1 | 90.6 | 5.9 | 1.19 | 0.90 |
| dalbavancin + | t = 1 mo | 4.1 | 90.1 | 6.1 | 1.45 | 0.43 |
| 0.6 mM CD + | t = 6 mo | 3.9 | 87.2 | 6.0 | 2.53 | 0.24 |
| 5% dextrose | | | | | | |
| 1 mg/mL | t = 0 | 3.9 | 87.0 | 5.6 | 1.18 | 0.88 |
| dalbavancin + | t = 1 mo | 3.9 | 90.0 | 6.2 | 1.47 | 0.40 |
| 5.5 mM CD | t = 6 mo | 3.8 | 85.0 | 8.9 | 2.53 | — |
| 1 mg/mL | t = 0 | 4.0 | 89.4 | 5.8 | 1.16 | 0.83 |
| dalbavancin + | t = 1 mo | 4.2 | 91.5 | 6.1 | 1.43 | 0.59 |
| 55 mM CD | t = 6 mo | 3.8 | 86.8 | 6.0 | 2.84 | 0.24 |
| 5 mg/mL | t = 0 | 4.1 | 92.0 | 5.9 | 1.15 | 0.87 |
| dalbavancin + | t = 1 mo | 4.1 | 90.1 | 6.1 | 1.52 | 0.57 |
| 55 mM CD | t = 6 mo | 3.6 | 84.5 | 8.9 | 3.02 | — |
| 5 mg/mL | t = 0 | 4.0 | 87.8 | 5.7 | 1.20 | 0.85 |
| dalbavancin + | t = 1 mo | 4.2 | 91.3 | 6.5 | 1.44 | 0.22 |
| 3.0 mM CD + | t = 6 mo | 3.5 | 85.0 | 9.0 | 2.42 | — |
| 5% dextrose | | | | | | |
| 1 mg/mL | t = 0 | 3.9 | 85.2 | 5.5 | 1.23 | 0.77 |
| dalbavancin + | t = 1 mo | 4.2 | 89.6 | 5.9 | 1.43 | 0.54 |
| 0.6 mM CD + | t = 6 mo | 3.8 | 86.9 | 6.2 | 2.57 | 0.34 |
| 10 mM Ca² | | | | | | |
| 1 mg/mL | t = 0 | 4.1 | 87.7 | 5.8 | 1.23 | 0.87 |
| dalbavancin + | t = 1 mo | 3.9 | 90.5 | 5.7 | 1.46 | 0.60 |
| 0.6 mM CD + | t = 6 mo | 3.8 | 86.8 | 6.2 | 2.60 | 0.18 |
| 10 mM Mg²⁺ | | | | | | |
| 5 mg/mL | t = 0 | 4.0 | 88.7 | 5.8 | 1.2 | 0.85 |
| dalbavancin + | t = 1 mo | 4.0 | 90.9 | 6.0 | 1.47 | 0.34 |
| 3.0 mM CD + | t = 6 mo | 3.6 | 85.0 | 8.9 | 2.53 | — |
| 10 mM Mg²⁺ | | | | | | |
| 1 mg/mL | t = 0 | 4.0 | 90.1 | 5.9 | 1.20 | 0.88 |
| dalbavancin + | t = 1 mo | 4.1 | 90.5 | 6.1 | 1.53 | 0.58 |
| 5.5 mM CD + | t = 6 mo | 3.8 | 86.8 | 6.0 | 2.83 | 0.37 |
| 2.0 mM Zn²⁺ | | | | | | |

TABLE 24
Stability of 1 mg/mL and 5 mg/mL dalbavancin formulations in 10 mM acetate
buffer, pH 4.5 including HPβCD, following extended storage at 40° C./75% RH

| | | | 1 | 2 | 3 | 6 |
|---|---|---|---|---|---|---|
| | Testing | T₀ | month | months | months | months |
| 1 mg/mL | Appearance | cl, co | cl, co | cl, co | cl, co | cl, co |
| dalbavancin + | pH | 4.44 | 4.47 | 4.39 | 4.46 | 4.47 |
| 0.6 mM CD | Assay % | 98.0 | 96.8 | 93.3 | 87.1 | 79.9 |
| | Chromatographic purity (%) | 97.93 | 95.75 | 93.01 | 90.02 | 82.70 |
| 1 mg/mL | Appearance | cl, co | cl, co | cl, co | cl, co | cl, co |
| dalbavancin + | pH | 4.48 | 4.48 | 4.40 | 4.47 | 4.47 |
| 0.6 mM CD + | Assay % | 100.4 | 96.8 | 93.5 | 85.2 | 80.4 |
| 5% dextrose | Chromatographic purity (%) | 97.94 | 95.41 | 92.89 | 90.09 | 82.96 |
| 1 mg/mL | Appearance | cl, co | cl, co | cl, co | cl, co | cl, co |
| dalbavancin + | pH | 4.63 | 4.58 | 4.58 | 4.63 | 4.69 |
| 5.5 mM CD | Assay % | 96.4 | 96.7 | 94.2 | 84.8 | 80.2 |
| | Chromatographic purity (%) | 97.94 | 95.40 | 92.43 | 89.17 | 81.98 |
| 1 mg/mL | Appearance | cl, co | cl, co | cl, co | cl, co | cl, co |
| dalbavancin + | pH | 4.50 | 4.47 | 4.45 | 4.50 | 4.50 |
| 55 mM CD | Assay % | 99.1 | 97.9 | 93.9 | 85.3 | 80.1 |
| | Chromatographic purity (%) | 98.01 | 95.17 | 91.79 | 88.50 | 80.50 |
| 5 mg/mL | Appearance | cl, co | cl, co | cl, co | cl, co | cl, co |
| dalbavancin + | pH | 4.36 | 4.34 | 4.35 | 4.42 | 4.57 |
| 55 mM CD | Assay % | 102.0 | 95.0 | 89.8 | 82.2 | 69.1 |
| | Chromatographic purity (%) | 97.98 | 94.37 | 90.55 | 86.85 | 78.70 |
| 5 mg/mL | Appearance | cl, co | cl, co | cl, co | cl, co | cl, co |
| dalbavancin + | pH | 4.37 | 4.35 | 4.27 | 4.41 | 4.31 |
| 3.0 mM CD + | Assay % | 97.4 | 97.7 | 92.6 | 87.0 | 71.6 |
| 5% dextrose | Chromatographic purity (%) | 97.95 | 95.76 | 95.76 | 90.35 | 82.98 |
| 1 mg/mL | Appearance | cl, co | cl, co | cl, co | cl, co | cl, co |
| dalbavancin + | pH | 4.53 | 4.55 | 4.44 | 4.52 | 4.46 |
| 0.6 mM CD + | Assay % | 94.6 | 96.4 | 93.0 | 85.0 | 78.3 |
| 10 mM Ca²⁺ | Chromatographic purity (%) | 97.90 | 95.66 | 92.71 | 89.57 | 81.93 |
| 1 mg/mL | Appearance | cl, co | cl, co | cl, co | cl, co | cl, co |
| dalbavancin + | pH | 4.56 | 4.56 | 4.47 | 4.52 | 4.50 |
| 0.6 mM CD + | Assay % | 96.7 | 97.5 | 98.8 | 86.2 | 79.6 |
| 10 mM Mg²⁺ | Chromatographic purity (%) | 97.90 | 96.82 | 92.40 | 90.27 | 82.38 |
| 5 mg/mL | Appearance | cl, co | cl, co | cl, co | cl, co | cl, co |
| dalbavancin + | pH | 4.43 | 4.48 | 4.35 | 4.37 | 4.38 |
| 3.0 mM CD + | Assay % | 98.5 | 96.3 | 91.1 | 84.4 | 68.9 |
| 10 mM Mg²⁺ | Chromatographic purity (%) | 97.95 | 95.45 | 92.43 | 89.27 | 81.43 |
| 1 mg/mL | Appearance | cl, co | cl, co | cl, co | cl, co | cl, co |
| dalbavancin + | pH | 4.62 | 4.57 | 4.54 | 4.57 | 4.57 |
| 5.5 mM CD + | Assay % | 99.9 | 97.3 | 93.7 | 85.3 | 80.1 |
| 2.0 mM Zn²⁺ | Chromatographic purity (%) | 97.92 | 95.40 | 92.14 | 89.20 | 81.51 |

Solution appearance (cl = clear; co = colorless; y = slight yellow color; p = slight pink color; sh = slightly hazy; h = hazy; ppt = precipitated)

TABLE 25
Chromatographic purity analysis of stability/purity
of 1 mg/ml and 5 mg/mL dalbavancin formulations in
acetate buffer, pH 4.5 including HPβCD at t =
0 and following extended storage at 40° C./75% RH for 1 mo

| | | dalbavancin component | | | % degradation | |
|---|---|---|---|---|---|---|
| | | distribution (%) | | | products | |
| | | A₀ + A₁ | B₀ | B₁ + B₂ | MAG | other |
| 1 mg/mL | t = 0 | 4.0 | 88.5 | 5.6 | 1.22 | 0.85 |
| dalbavancin + | t = 1 mo | 3.8 | 87.5 | 5.6 | 3.93 | 0.32 |
| 0.6 mM CD | | | | | | |
| 1 mg/mL | t = 0 | 4.1 | 90.6 | 5.9 | 1.19 | 0.90 |
| dalbavancin + | t = 1 mo | 3.9 | 87.2 | 5.7 | 3.99 | 0.60 |
| 0.6 mM CD + | | | | | | |
| 5% dextrose | | | | | | |
| 1 mg/mL | t = 0 | 3.9 | 87.0 | 5.6 | 1.18 | 0.88 |
| dalbavancin + | t = 1 mo | 3.9 | 87.2 | 5.6 | 4.27 | 0.33 |
| 5.5 mM CD | | | | | | |
| 1 mg/mL | t = 0 | 4.0 | 89.4 | 5.8 | 1.16 | 0.83 |
| dalbavancin + | t = 1 mo | 4.2 | 88.1 | 5.6 | 4.60 | 0.23 |
| 55 mM CD | | | | | | |
| 5 mg/mL | t = 0 | 4.1 | 92.0 | 5.9 | 1.15 | 0.87 |
| dalbavancin + | t = 1 mo | 3.7 | 85.4 | 5.9 | 9.45 | — |
| 55 mM CD | | | | | | |
| 5 mg/mL | t = 0 | 4.0 | 87.8 | 5.7 | 1.20 | 0.85 |
| dalbavancin + | t = 1 mo | 3.9 | 87.6 | 6.1 | 3.92 | 0.32 |
| 3.0 mM CD + | | | | | | |
| 5% dextrose | | | | | | |
| 1 mg/mL | t = 0 | 3.9 | 85.2 | 5.5 | 1.23 | 0.77 |
| dalbavancin + | t = 1 mo | 3.9 | 86.6 | 5.9 | 4.03 | 0.31 |
| 0.6 mM CD + | | | | | | |
| 10 mM Ca²⁺ | | | | | | |
| 1 mg/mL | t = 0 | 4.1 | 87.7 | 5.8 | 1.23 | 0.87 |
| dalbavancin + | t = 1 mo | 4.1 | 87.7 | 5.8 | 4.00 | 0.18 |
| 0.6 mM CD + | | | | | | |
| 10 mM Mg²⁺ | | | | | | |
| 5 mg/mL | t = 0 | 4.0 | 88.7 | 5.8 | 1.2 | 0.85 |
| dalbavancin + | t = 1 mo | 4.0 | 86.7 | 5.8 | 7.57 | — |
| 3.0 mM CD + | | | | | | |
| 10 mM Mg²⁺ | | | | | | |
| 1 mg/mL | t = 0 | 4.0 | 90.1 | 5.9 | 1.20 | 0.88 |
| dalbavancin + | t = 1 mo | 3.9 | 87.7 | 5.8 | 4.38 | 0.23 |
| 5.5 mM CD + | | | | | | |
| 2.0 mM Zn²⁺ | | | | | | |

Following storage at 25° C./60% RH, all formulations were stable by all measures (Tables 26 and 27). The levels of B₀ relative to the other components were similar to the starting levels of B₀ (high 80%-low 90%). The levels of MAG impurity were somewhat increased relative to the initial MAG levels and were two times those of the 5° C. storage conditions, but in all cases still <3% by chromatographic area for at least 6 months. Low levels of a degradant impurity, DB-R6 (RRT 1.26) was observed for only the formulation comprising CD and dextrose without metal ions and was not present in the metal-containing formulations.

A notable difference was observed in comparing the formulations without CD and those with CD following storage at 25° C./60% RH. For the 1 mg/mL and 5 mg/mL formulations comprising metal ions but no CD, B₀ made up less than 90% of the total dalbavancin component. MAG levels were all higher than 1.5% following 25° C./60% RH storage. The addition of CD to the lower concentration formulations notably increased the B₀ component (all at or about 90%) and decreased the MAG impurity level (all <1.5%). The overall level of impurities as also improved in the CD-containing formulations (<2.6% in all cases) as compared to the formulations not including CD (2.5-3+%). These improvements were even further emphasized for the lower concentration formulations stored at 40° C./75% RH for 1 mo. Whereas several of the formulations containing metal ions only failed one or more of the chromatographic purity tests after 40° C./75% RH storage (i.e. 5 mg/mL dalbavancin+10 mM Ca²⁺ and 1 mg/mL+10 mM Mg²⁺), all of the CD-containing formulations passed all tests following the same storage conditions. As a direct comparison, the formulation comprising 1 mg/mL dalbavancin+10 mM Mg²⁺ had a B₀ component percentage of 80.8, and overall purity of 88.8%, and a MAG level of 9.28%. The addition of 0.6 mM CD improved the overall purity to greater than 97%, maintained the B₀ component at a level of 87.7% and had 4.0% by area MAG present. Overall, the CD-containing formulations had MAG levels of less than 5% in all cases and overall purity of at least 94% in all cases. Further, as seen in Table 25, several of the CD+ion-containing formulations were stable at least 2 months following storage at 40° C./75% RH. Overall, it is clear that the inclusion of cyclodextrin surprisingly improves dalbavancin stability under high temperature storage conditions.

### Example 4—Influence of Cyclodextrin on the Stability of Dalbavancin Formulations in Phosphate Buffer

As noted in Example 2, a dalbavancin in phosphate buffer could not be formulated without significant precipitation of the active ingredient. However, it was surprisingly found that the addition of CD to the phosphate-buffered formulation not only resulted in formulations without precipitation, but that the formulations were also highly stable following extended storage at both 5° C. and 25° C./65% RH. The following Tables provide stability data for dalbavancin formulations of higher (20 mg/mL) and lower (1 mg/mL and 5 mg/mL) dalbavancin formulations in phosphate buffer, variously including combinations of Mg²⁺, Ca²⁺, Zn²⁺ and CD.

TABLE 30
Stability of 20 mg/mL dalbavancin formulations in 10 mM phosphate buffer,
pH 7.0 including HPβCD, following extended storage at 5° C.

| | | | 1 | 2 | 3 | 6 |
|---|---|---|---|---|---|---|
| | Testing | T₀ | month | months | months | months |
| +55 mM CD | Appearance | cl, co | co, sh | co, sh | co, sh | p, sh |
| | pH | 7.03 | 7.03 | 7.02 | 6.94 | 6.97 |
| | Assay % | 103.0 | 101.2 | 100.1 | 96.2 | 98.6 |
| | Chromatographic purity (%) | 97.96 | 98.26 | 98.80 | 98.76 | 98.77 |
| +110 mM CD | Appearance | cl, co | cl, co | cl, co | co, sh | p, cl |
| | pH | 7.08 | 6.98 | 7.17 | 7.05 | 7.09 |
| | Assay % | 99.8 | 99.1 | 99.5 | 94.3 | 97.6 |
| | Chromatographic purity (%) | 97.96 | 98.23 | 98.74 | 98.73 | 98.74 |
| +55 mM CD + | Appearance | cl, co | co, h | co, h | co, h | p, cl |
| 50 mM Mg²⁺ | pH | 7.10 | 7.00 | 7.05 | 6.95 | 6.98 |
| | Assay % | 98.1 | 98.8 | 102.4 | 95.1 | 96.6 |
| | Chromatographic purity (%) | 97.96 | 98.17 | 98.78 | 98.71 | 98.71 |
| +110 mM CD + | Appearance | cl, co | y, h | y, h | ho, h | p, cl |
| 50 mM Mg²⁺ | pH | 6.99 | 6.75 | 6.92 | 6.82 | 6.65 |
| | Assay % | 98.8 | 101.9 | 99.2 | 94.5 | 97.1 |
| | Chromatographic purity (%) | 97.94 | 98.13 | 98.87 | 98.51 | 98.67 |

Solution appearance (cl = clear; co = colorless; y = slight yellow color; p = slight pink color; sh = slightly hazy; h = hazy; ppt = precipitated)

TABLE 31
Chromatographic purity analysis of stability/purity
of 20 mg/mL dalbavancin formulations in 10 mM phosphate
buffer, pH 7.0 including HPβCD, following extended
storage at 5° C. at t = 0, 1 month and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| +55 mM CD | t = 0 | 4.1 | 92.7 | 6.1 | 1.20 | 0.84 |
| | t = 1 mo | 4.0 | 91.2 | 6.1 | 1.20 | 0.54 |
| | t = 6 mo | 4.0 | 88.4 | 6.4 | 1.23 | — |
| +110 mM CD | t = 0 | 4.0 | 88.9 | 5.9 | 1.2 | 0.84 |
| | t = 1 mo | 3.9 | 89.2 | 6.0 | 1.21 | 0.56 |
| | t = 6 mo | 3.9 | 88.2 | 6.4 | 1.26 | — |
| +55 mM CD + | t = 0 | 4.0 | 88.4 | 5.8 | 1.21 | 0.85 |
| 50 mM Mg²⁺ | t = 1 mo | 3.8 | 89.0 | 6.0 | 1.20 | 0.63 |
| | t = 6 mo | 3.9 | 88.2 | 6.4 | 1.29 | — |
| +110 mM CD + | t = 0 | 4.0 | 89.0 | 5.8 | 1.22 | 0.84 |
| 50 mM Mg²⁺ | t = 1 mo | 4.1 | 91.9 | 5.9 | 1.26 | 0.61 |
| | t = 6 mo | 4.0 | 88.1 | 6.4 | 1.33 | — |

TABLE 32
Stability of 20 mg/mL dalbavancin formulations in 10 mM phosphate buffer, pH 7.0 including
HPβCD, following extended storage at 25° C./60% RH for 0, 1, 2, 3 and 6 months

| | | | 1 | 2 | 3 | 6 |
|---|---|---|---|---|---|---|
| | Testing | T₀ | month | months | months | months |
| +55 mM CD | Appearance | cl, co | co, sh | co, sh | p, sh | p, sh |
| | pH | 7.03 | 6.93 | 7.14 | 6.85 | 6.92 |
| | Assay % | 103.0 | 95.0 | 91.1 | 88.5 | 84.0 |
| | Chromatographic purity (%) | 97.96 | 97.29 | 96.58 | 95.50 | 94.94 |
| +110 mM CD | Appearance | cl, co | co, sh | co, sh | y, h | cl, p |
| | pH | 7.08 | 6.89 | 7.11 | 6.92 | 6.90 |
| | Assay % | 99.8 | 96.3 | 93.9 | 86.5 | 84.4 |
| | Chromatographic purity (%) | 97.96 | 97.35 | 96.70 | 94.75 | 94.55 |
| +55 mM CD + | Appearance | cl, co | co, h | co, h | h, p | cl, lp |
| 50 mM Mg²⁺ | pH | 7.10 | 6.87 | 7.01 | 6.81 | 6.73 |
| | Assay % | 98.1 | 94.7 | 91.7 | 84.6 | 84.4 |
| | Chromatographic purity (%) | 97.96 | 97.01 | 96.55 | 95.07 | 94.64 |
| +110 mM CD + | Appearance | cl, co | co, h | co, h | co, h | cl, lp |
| 50 mM Mg²⁺ | pH | 6.99 | 6.66 | 6.80 | 6.59 | 6.55 |
| | Assay % | 98.8 | 97.4 | 94.0 | 87.9 | 86.1 |
| | Chromatographic purity (%) | 97.94 | 97.52 | 96.97 | 95.51 | 94.69 |

Solution appearance (cl = clear; co = colorless; y = slight yellow color; p = slight pink color; sh = slightly hazy; h = hazy; ppt = precipitated)

TABLE 33
Chromatographic purity analysis of stability/purity of 20 mg/mL
dalbavancin formulations in phosphate buffer, pH 7.0 following
extended storage at 25° C./60% RH for 0, 1 and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| +55 mM CD | t = 0 | 4.1 | 92.7 | 6.1 | 1.20 | 0.84 |
| | t = 1 mo | 4.0 | 86.4 | 5.6 | 1.39 | 1.32 |
| | t = 6 mo | 3.9 | 85.6 | 5.2 | 2.56 | 2.5 |
| +110 mM CD | t = 0 | 4.0 | 88.9 | 5.9 | 1.2 | 0.84 |
| | t = 1 mo | 4.0 | 86.7 | 5.6 | 1.47 | 1.23 |
| | t = 6 mo | 3.9 | 85.4 | 5.0 | 2.61 | 2.84 |
| +55 mM CD + | t = 0 | 4.0 | 88.4 | 5.8 | 1.21 | 0.85 |
| 50 mM Mg²⁺ | t = 1 mo | 3.9 | 85.3 | 5.5 | 1.47 | 1.52 |
| | t = 6 mo | 3.9 | 85.5 | 5.0 | 2.53 | 2.83 |
| | t = 0 | 4.0 | 89.0 | 5.8 | 1.22 | 0.84 |
| +110 mM CD + | t = 1 mo | 3.9 | 87.8 | 5.7 | 1.53 | 0.95 |
| 50 mM Mg²⁺ | t = 6 mo | 4.0 | 85.5 | 5.0 | 2.78 | 2.53 |

TABLE 34
Stability of 20 mg/mL dalbavancin formulations in 10 mM phosphate buffer, pH
7.0 including HPβCD, following extended storage at 40° C./75% RH

| | | | 1 | 2 | 3 | 6 |
|---|---|---|---|---|---|---|
| | Testing | T₀ | month | months | months | months |
| +55 mM CD | Appearance | cl, co | y, h | y, h | y, h | y, ppt |
| | pH | 7.03 | 6.87 | 7.05 | 6.80 | 6.73 |
| | Assay % | 103.0 | 66.8 | 53.9 | 35.6 | 29.0 |
| | Chromatographic purity (%) | 97.96 | 89.56 | 81.13 | 77.72 | 76.86 |
| +110 mM CD | Appearance | cl, co | cl, y | y, h | y, h | y, ppt |
| | pH | 7.08 | 6.99 | 7.14 | 6.89 | 6.77 |
| | Assay % | 99.8 | 70.3 | 50.2 | 40.1 | 24.9 |
| | Chromatographic purity (%) | 97.96 | 88.79 | 77.80 | 73.81 | 73.81 |
| +55 mM CD + | Appearance | cl, co | co, h | y, h | y, h | y, ppt |
| 50 mM Mg²⁺ | pH | 7.10 | 6.70 | 6.85 | 6.68 | 6.63 |
| | Assay % | 98.1 | 79.1 | 66.2 | 55.1 | 35.8 |
| | Chromatographic purity (%) | 97.96 | 91.88 | 84.75 | 73.81 | 76.71 |
| +110 mM CD + | Appearance | cl, co | co, h | y, h | y, h | y, ppt |
| 50 mM Mg²⁺ | pH | 6.99 | 6.69 | 6.84 | 6.67 | 6.55 |
| | Assay % | 98.8 | 80.0 | 67.7 | 57.2 | 38.7 |
| | Chromatographic purity (%) | 97.94 | 91.17 | 83.61 | 77.74 | 74.52 |

Solution appearance (cl = clear; co = colorless; y = slight yellow color; p = slight pink color; sh = slightly hazy; h = hazy; ppt = precipitated)

Overall, the dalbavancin formulations in phosphate buffer were highly stable following storage of at least 6 months under refrigerated conditions. They demonstrated greater than 98% chromatographic purity and contained less than 2% of MAG. Further, the levels of the B₀ component remained high following 6 months of storage, greater than 88% in all cases. Following 6 months of storage at 25° C., higher levels of dalbavancin degradation (other impurities present included DB-RS and DB-R6) were observed than were observed for the acetate formulations after 6 months, but the formulations were still highly stable for at least three months. Like many of the formulations tested, the formulations were not highly stable following storage at 40° C., showing significant degradation at even the 1-month mark. Notably, following 40° C. storage, a number of unidentified impurities were observed, having RRT of 0.85, 1.42, 1.45, 1.48, and 1.52. Levels of these impurities varied between 0 and 2.5% (chromatographic area).

TABLE 35
Stability of 1 mg/mL and 5 mg/mL dalbavancin formulations
in 10 mM phosphate buffer, pH 7.0 including HPβCD following
extended storage at 5° C. of 0, 1, 2, 3, and 6 months

| | | | 1 | 2 | 3 | 6 |
|---|---|---|---|---|---|---|
| | Testing | T₀ | month | months | months | months |
| 1 mg/mL | Appearance | cl, co | cl, co | cl, co | cl, co | cl, co |
| dalbavancin + | pH | 7.03 | 6.88 | 6.96 | 6.87 | 6.93 |
| 5.5 mM CD | Assay % | 96.3 | 101.1 | 101.1 | 97.2 | 99.6 |
| | Chromatographic purity (%) | 97.95 | 98.58 | 98.76 | 98.76 | 98.66 |
| 1 mg/mL | Appearance | cl, co | cl, co | cl, co | cl, co | cl, co |
| dalbavancin + | pH | 6.97 | 6.96 | 6.97 | 6.94 | 6.93 |
| 55 mM CD | Assay % | 99.2 | 101.9 | 101.9 | 97.2 | 99.6 |
| | Chromatographic purity (%) | 98.03 | 98.51 | 98.77 | 98.80 | 98.61 |
| 5 mg/mL | Appearance | cl, co | cl, co | cl, co | cl, co | cl, co |
| dalbavancin + | pH | 7.06 | 6.96 | 7.01 | 6.94 | 6.96 |
| 27.5 mM CD | Assay % | 96.6 | 101.7 | 99.7 | 96.1 | 89.7 |
| | Chromatographic purity (%) | 98.03 | 98.39 | 98.39 | 98.79 | 98.87 |
| 5 mg/mL | Appearance | cl, co | cl, co | cl, co | cl, co | cl, co |
| dalbavancin + | pH | 7.03 | 6.92 | 6.99 | 6.86 | 6.94 |
| 55 mM CD | Assay % | 99.1 | 101.8 | 99.7 | 96.5 | 87.1 |
| | Chromatographic purity (%) | 98.0 | 98.39 | 97.62 | 98.78 | 98.86 |
| 1 mg/mL | Appearance | cl, co | cl, co | cl, co | cl, co | cl, co |
| dalbavancin + | pH | 7.05 | 6.91 | 6.95 | 6.89 | 6.92 |
| 5.5 mM CD + | Assay % | 95.7 | 100.5 | 100.9 | 95.6 | 98.8 |
| 5% dextrose | Chromatographic purity (%) | 98.04 | 98.62 | 98.70 | 98.53 | 98.89 |
| 5 mg/mL | Appearance | cl, co | cl, co | cl, co | cl, co | cl, co |
| dalbavancin + | pH | 7.02 | 6.90 | 6.96 | 6.88 | 6.81 |
| 27.5 mM CD + | Assay % | 95.8 | 99.1 | 98.9 | 94.2 | 89.2 |
| 5% dextrose | Chromatographic purity (%) | 98.02 | 98.27 | 97.69 | 98.49 | 98.85 |
| 1 mg/mL | Appearance | cl, co | cl, co | cl, co | cl, co | cl, co |
| dalbavancin + | pH | 7.01 | 6.87 | 6.86 | 6.86 | 6.46 |
| 5.5 mM CD + | Assay % | 97.5 | 101.3 | 101.5 | 96.1 | 99.9 |
| 50 mM Mg²⁺ | Chromatographic purity (%) | 97.92 | 98.50 | 98.89 | 98.70 | 98.64 |
| 1 mg/mL | Appearance | cl, co | cl, co | cl, co | cl, co | cl, co |
| dalbavancin + | pH | 7.01 | 6.86 | 6.87 | 6.94 | 6.76 |
| 55 mM CD + | Assay % | 99.0 | 101.9 | 102.2 | 97.3 | 100.2 |
| 50 mM Mg²⁺ | Chromatographic purity (%) | 97.04 | 98.51 | 98.71 | 98.71 | 98.72 |
| 5 mg/mL | Appearance | cl, co | cl, co | cl, co | cl, co | cl, co |
| dalbavancin + | pH | 7.03 | 6.92 | 6.99 | 6.86 | 6.94 |
| 55 mM CD + | Assay % | 97.6 | 99.8 | 99.4 | 99.5 | 90.7 |
| 50 mM Mg²⁺ | Chromatographic purity (%) | 97.98 | 98.22 | 97.66 | 97.66 | 97.66 |
| 5 mg/mL | Appearance | cl, co | co, ppt | co, ppt | co, ppt | co, ppt |
| dalbavancin + | pH | 7.04 | 6.69 | 6.43 | 6.89 | 6.62 |
| 27.5 mM CD + | Assay % | 94.1 | 98.8 | 97.9 | 94.6 | 91.7 |
| 50 mM Mg²⁺ | Chromatographic purity (%) | 98.03 | 98.20 | 98.61 | 98.64 | 98.66 |

Solution appearance (cl = clear; co = colorless; y = slight yellow color; p = slight pink color; sh = slightly hazy; h = hazy; ppt = precipitated)

TABLE 36
Chromatographic purity analysis of stability/purity
of 1 mg/ml and 5 mg/mL dalbavancin formulations in
10 mM phosphate buffer, pH 7.0 including HPβCD following
extended storage at 5° C. of 0, 1 and 6 months

| | | dalbavancin component | | | % degradation | |
|---|---|---|---|---|---|---|
| | | distribution (%) | | | products | |
| | | A₀ + A₁ | B₀ | B₁ + B₂ | MAG | other |
| 1 mg/mL | t = 0 | 3.9 | 86.8 | 5.6 | 1.19 | 0.91 |
| dalbavancin + | t = 1 mo | 4.0 | 91.0 | 6.0 | 1.19 | 0.44 |
| 5.5 mM CD | t = 6 mo | 3.9 | 88.2 | 6.5 | 1.16 | 0.18 |
| 1 mg/mL | t = 0 | 4.0 | 89.5 | 5.8 | 1.12 | 0.85 |
| dalbavancin + | t = 1 mo | 4.0 | 91.6 | 6.1 | 1.17 | 0.32 |
| 55 mM CD | t = 6 mo | 3.9 | 88.2 | 6.5 | 1.18 | 0.21 |
| 5 mg/mL | t = 0 | 4.0 | 89.0 | 5.7 | 1.12 | 0.85 |
| dalbavancin + | t = 1 mo | 4.4 | 91.3 | 6.1 | 1.17 | 0.44 |
| 27.5 mM CD | t = 6 mo | 3.7 | 86.0 | 9.2 | 1.13 | — |
| 5 mg/mL | t = 0 | 4.0 | 89.2 | 5.7 | 1.10 | 0.85 |
| dalbavancin + | t = 1 mo | 4.1 | 91.6 | 6.1 | 1.17 | 0.44 |
| 55 mM CD | t = 6 mo | 3.7 | 86.1 | 9.1 | 1.14 | — |
| 1 mg/mL | t = 0 | 3.8 | 86.4 | 5.6 | 1.17 | 0.80 |
| dalbavancin + | t = 1 mo | 4.1 | 90.2 | 6.1 | 1.17 | 0.33 |
| 5.5 mM CD + | t = 6 mo | 3.9 | 88.4 | 6.6 | 1.09 | — |
| 5% dextrose | | | | | | |
| 5 mg/mL | t = 0 | 3.9 | 86.5 | 5.4 | 1.17 | 0.81 |
| dalbavancin + | t = 1 mo | 4.1 | 89.1 | 6.0 | 1.20 | 0.53 |
| 27.5 mM CD + | t = 6 mo | 3.7 | 86.0 | 9.1 | 1.15 | — |
| 5% dextrose | | | | | | |
| 1 mg/mL | t = 0 | 4.0 | 87.8 | 5.7 | 1.21 | 0.87 |
| dalbavancin + | t = 1 mo | 4.1 | 91.3 | 6.0 | 1.25 | 0.25 |
| 5.5 mM CD + | t = 6 mo | 3.9 | 88.1 | 6.5 | 1.28 | 0.18 |
| 50 mM Mg²⁺ | | | | | | |
| 1 mg/mL | t = 0 | 4.0 | 89.2 | 5.7 | 1.17 | 0.91 |
| dalbavancin + | t = 1 mo | 4.3 | 91.4 | 6.2 | 1.17 | 0.32 |
| 55 mM CD + | t = 6 mo | 4.0 | 88.3 | 6.5 | 1.28 | — |
| 50 mM Mg²⁺ | | | | | | |
| 5 mg/mL | t = 0 | 4.0 | 88.0 | 5.7 | 1.16 | 0.86 |
| dalbavancin + | t = 1 mo | 4.0 | 89.9 | 5.9 | 1.23 | 0.55 |
| 55 mM CD + | t = 6 mo | 4.3 | 88.5 | 6.0 | 1.24 | 1.10 |
| 50 mM Mg²⁺ | | | | | | |
| 5 mg/mL | t = 0 | 3.7 | 84.9 | 5.5 | 1.24 | 0.73 |
| dalbavancin + | t = 1 mo | 3.8 | 89.0 | 6.0 | 1.25 | 0.55 |
| 27.5 mM CD + | t = 6 mo | 3.7 | 86.0 | 9.1 | 1.24 | — |
| 50 mM Mg²⁺ | | | | | | |

As seen from Tables 35 and 36, the lower concentration dalbavancin formulations in phosphate buffer were highly stable when stored under refrigerated conditions. They exhibited high levels of pH control out to at least 6 months of storage. Phosphate-buffered formulations having lower concentrations of dalbavancin that were stored at 0° C. all had less than 2% of the problematic MAG impurity up to at least 6 months of storage while maintaining at least 86% of the B₀ component and high levels of overall chromatographic purity. Both the unknown impurity having RRT=0.85 and/or the unknown impurity having RRT=0.92 were present in some of the formulations (indicated as “other”).

TABLE 37
Stability of 1 mg/mL and 5 mg/mL dalbavancin formulations in 10 mM phosphate
buffer, pH 7.0 including HPβCD following extended storage at 25° C./60% RH

| | | | 1 | 2 | 3 | 6 |
|---|---|---|---|---|---|---|
| | Testing | T₀ | month | months | months | months |
| 1 mg/mL | Appearance | cl, co | cl, co | cl, co | cl, co | cl, co |
| dalbavancin + | pH | 7.03 | 6.87 | 6.86 | 6.84 | 6.86 |
| 5.5 mM CD | Assay % | 96.3 | 98.1 | 96.7 | 91.8 | 89.0 |
| | Chromatographic purity (%) | 97.95 | 97.44 | 96.69 | 94.71 | 94.80 |
| 1 mg/mL | Appearance | cl, co | cl, co | cl, co | cl, co | cl, co |
| dalbavancin + | pH | 6.97 | 6.89 | 6.86 | 6.82 | 6.87 |
| 55 mM CD | Assay % | 99.2 | 98.9 | 97.2 | 93.5 | 90.0 |
| | Chromatographic purity (%) | 98.03 | 97.34 | 96.50 | 94.88 | 94.61 |
| 5 mg/mL | Appearance | cl, co | co, ppt | co, ppt | co, ppt | co, ppt |
| dalbavancin + | pH | 7.06 | 6.41 | 6.87 | 6.79 | 6.85 |
| 27.5 mM CD | Assay % | 96.6 | 99.5 | 96.0 | 90.1 | 80.4 |
| | Chromatographic purity (%) | 98.03 | 97.06 | 96.45 | 95.86 | 95.29 |
| 5 mg/mL | Appearance | cl, co | co, ppt | co, ppt | co, ppt | co, ppt |
| dalbavancin + | pH | 7.03 | 6.87 | 6.86 | 6.85 | 6.86 |
| 55 mM CD | Assay % | 99.1 | 104.9 | N/A | 89.6 | 78.7 |
| | Chromatographic purity (%) | 98.0 | 97.15 | N/A | 95.85 | 95.29 |
| 1 mg/mL | Appearance | cl, co | cl, co | cl, co | cl, co | cl, co |
| dalbavancin + | pH | 7.05 | 6.63 | 6.36 | 6.26 | 6.03 |
| 5.5 mM CD + | Assay % | 95.7 | 97.1 | 95.0 | 92.1 | 90.3 |
| 5% dextrose | Chromatographic purity (%) | 98.04 | 96.95 | 96.66 | 94.38 | 94.69 |
| 5 mg/mL | Appearance | cl, co | co, ppt | co, ppt | co, ppt | co, ppt |
| dalbavancin + | pH | 7.02 | 6.70 | 6.64 | 6.82 | 6.43 |
| 27.5 mM CD + | Assay % | 95.8 | 96.7 | 96.0 | 92.6 | 83.8 |
| 5% dextrose | Chromatographic purity (%) | 98.02 | 97.15 | 96.34 | 94.51 | 94.79 |
| 1 mg/mL | Appearance | cl, co | cl, co | cl, co | cl, co | cl, co |
| dalbavancin + | pH | 7.01 | 7.03 | 6.26 | 6.31 | 6.26 |
| 5.5 mM CD + | Assay % | 97.5 | 100.0 | 99.4 | 93.5 | 95.4 |
| 50 mM Mg²⁺ | Chromatographic purity (%) | 97.92 | 97.44 | 96.11 | 96.55 | 95.91 |
| 1 mg/mL | Appearance | cl, co | cl, co | cl, co | cl, co | cl, co |
| dalbavancin + | pH | 7.01 | 6.55 | 6.49 | 6.49 | 6.38 |
| 55 mM CD + | Assay % | 99.0 | 99.1 | 96.4 | 96.3 | 93.8 |
| 50 mM Mg²⁺ | Chromatographic purity (%) | 97.04 | 97.31 | 98.71 | 95.58 | 95.43 |
| 5 mg/mL | Appearance | cl, co | cl, co | cl, co | cl, co | cl, co |
| dalbavancin + | pH | 7.03 | 6.66 | 6.56 | 6.49 | 6.48 |
| 55 mM CD + | Assay % | 97.6 | 99.3 | 95.6 | 93.8 | 82.4 |
| 50 mM Mg²⁺ | Chromatographic purity (%) | 97.98 | 97.33 | 97.33 | 95.75 | 95.44 |
| 5 mg/mL | Appearance | cl, co | co, ppt | co, ppt | co, ppt | co, ppt |
| dalbavancin + | pH | 7.04 | 6.70 | 6.64 | 6.82 | 6.43 |
| 27.5 mM CD + | Assay % | 94.1 | 97.1 | 94.9 | 89.1 | 84.0 |
| 50 mM Mg²⁺ | Chromatographic purity (%) | 98.03 | 97.35 | 97.5 | 95.77 | 95.95 |

Solution appearance (cl = clear; co = colorless; y = slight yellow color; p = slight pink color; sh = slightly hazy; h = hazy; ppt = precipitated)

Table 37 shows the stability data for the lower (1 mg/mL and 5 mg/mL) concentration dalbavancin formulations following 0, 1, 2, 3 and 6 months of storage at 25° C./60% RH. Compared to the refrigerated formulations (Table 35), the formulations stored at room temperature showed less pH control, although all were still within acceptable limits. The purity levels as determined by both chromatography methods high levels of stability to at least 3 months, but some of the formulations (e.g. 5 mg/mL dalbavancin+55 mM CD+50 mM Mg²⁺) showed degradation of the dalbavancin at the 6 month time point by the chromatography assays. Chromatography assays showed the presence of several impurities, including those having DB-R2, DB-R6 and unknown impurities at RRT=0.39, 0.85, and 1.45, present even at the 1-month time period (data not shown).

Dalbavancin formulations identical to those above (10 mM phosphate buffer, pH 7.0 including HPβCD) following extended storage at 40° C./75% RH were also monitored. Even at the 1-month time point, some of the formulations stored at 40° C. showed evidence of precipitation and nearly every sample showed precipitation by the 2-month time point. The pH values of the formulations showed considerable variance, some falling well outside of acceptable ranges, and nearly every formulation failed the chromatographic assays at the 1-month time point, all formulations were outside of the acceptable ranges by chromatography by the 2-month time point (data not shown).

### Comparative Example 1

In order to understand how the formulations of the disclosure compared to the currently marketed lyophilized product, a comparative study was undertaken to compare the stability of lyophililzed dalbavancin in water compared to the inventive formulations. A 20 mg/mL solution of lyophilized dalbavancin in water as stored at 25° C. for 24 h and 48 h. The stability results of dalbavancin are shown in Table 38.

TABLE 38
Comparative Example 1

| | | | | | | % degradation | |
|---|---|---|---|---|---|---|---|
| | | | dalbavancin component | | | products | |
| | | Assay | distribution (%) | | | | Total |
| | pH | % | A₀ + A₁ | B₀ | B₁ + B₂ | MAG | impurities |
| RLD | | 103.1 | 3.85 | 82.81 | 7.89 | 0.95 | 5.32 |
| t = 0 hrs | | | | | | | |
| RLD | | 103.0 | 4.06 | 83.58 | 7.99 | 1.22 | 4.35 |
| t = 24 hrs | | | | | | | |
| RLD | 4.40 | 93.3 | 4.00 | 83.46 | 7.83 | 1.25 | 4.68 |
| t = 48 hrs | | | | | | | |

As seen in Table 38, after only 48 h of storage at 25° C., the dalbavancin in water showed a marked decrease in the purity of the sample, with only 90% percent dalbavancin (total content) remaining and significantly higher levels of MAG apparent as compared to the inventive formulations.

### Comparative Example 2

The stability of samples of dalbavancin in water alone was measured to understand how the presence of buffer affects stability. In all cases, 20 mg/mL dalbavancin was dissolved in sterile water with or without pH adjustment. For the samples with pH adjustment, the pH was adjusted to 4.5 using 0.1 N NaOH. All samples were store at different temperatures for 1 month.

TABLE 39
Comparative Example 2

| | | visual | pH | Assay % | A₀ + A₁/B₀/B₁ + B₂ | MAG |
|---|---|---|---|---|---|---|
| H₂O (no pH | (t = 0) | cl, co | 3.50 | 106.1 | 1.5/94.6/3.2 | 0.68 |
| adjustment) | 5° C. (t = 1 mo) | cl, co | 3.67 | 103.3 | 1.4/92.1/5.9 | 0.66 |
| | 25° C. (t = 1 mo) | cl, co | 3.70 | 102.1 | 1.4/90.3/5.8 | 2.58 |
| | 40° C. (t = 1 mo) | cl, co | 3.82 | 83.4 | 1.2/78.5/4.7 | 15.53 |
| | (t = 0) | cl, co | 4.54 | 105.0 | 1.5/94.5/3.3 | 0.66 |
| H₂O (pH | 5° C. (t = 1 mo) | cl, co | 4.59 | 104.9 | 1.4/92.2/5.8 | 0.63 |
| adjusted) | 25° C. (t = 1 mo) | cl, co | 4.63 | 101.1 | 1.4/91.4/5.7 | 1.51 |
| | 40° C. (t = 1 mo) | cl, co | 4.71 | 91.3 | 1.4/84.8/5.1 | 8.64 |

Based on the data in Table 39, it is clear that water alone is insufficient to stabilize dalbavancin. After 1 month of storage at 5° C. in water alone, the pH of the dalbavancin solution rose from 3.50 to 3.67. When stored under room temperature or hot conditions, the pH rose to 3.70 and 3.82, respectively. Similar behavior was seen for the pH adjusted water solutions. From an initial pH of 4.54, the solutions stored at 5° C., 25° C. and 40° C. rose 0.05, 0.09 and 0.17 pH units, respectively. Likewise, the water only solutions showed higher levels of MAG and lower levels of dalbavancin, particularly the B₀ component, than their buffered counterparts following the same storage time. While the pH adjusted solutions of dalbavancin faired better than the unadjusted solutions (see e.g. the pH adjusted solution stored at 40° C. for 1 month showed 91.3% dalbavancin remaining as compared to just 83.4% for the unadjusted sample under the same storage conditions), they are still not expected to be sufficiently stable over long periods of time.

Many variations and modifications may be made to the above-described embodiments of the disclosure without departing substantially from the spirit and various principles of the disclosure. All such modifications and variations are intended to be included herein within the scope of this disclosure and protected by the following claims.

What is claimed is:

1. An aqueous pharmaceutical composition for intravenous administration comprising:
i) dalbavancin or a pharmaceutically acceptable salt thereof;
ii) a pharmaceutically acceptable buffer having a pH of from about 4.0 to about 8.0; and
iii) one or more of a pharmaceutically acceptable divalent salt and/or a pharmaceutically acceptable cyclodextrin, wherein the divalent salt is distinct from the buffer, and
wherein the pharmaceutical composition retains about 90% or more of the initial concentration of dalbavancin or pharmaceutically acceptable salt thereof after storage for at least six months when stored at about 5° C.

2. The pharmaceutical composition of claim 1 wherein the dalbavancin is dalbavancin hydrochloride.

3. The pharmaceutical composition of claim 1 having a pH of about 4.0 to about 5.0.

4. The pharmaceutical composition of claim 1, wherein the buffer is an acetate buffer.

5. The pharmaceutical composition claim 1 having a pH of about 6.5 to about 7.5.

6. The pharmaceutical composition of claim 1, wherein the buffer is a phosphate buffer.

7. The pharmaceutical composition of claim 1, wherein the buffer has a concentration of from about 1 to about 20 mM.

8. The pharmaceutical composition of claim 1, wherein the formulation comprises the divalent salt.

9. The pharmaceutical composition of claim 8, wherein a cation of the divalent salt is selected from the group consisting of Ca²⁺, Mg²⁺, Zn²⁺ and mixtures thereof.

10.-13. (canceled)

14. The pharmaceutical composition of claim 8, wherein the divalent cation is present in an amount of from about 0.1 mM to about 50 mM.

15. The pharmaceutical composition of claim 14, wherein the divalent cation is present in an amount of from about 1 mM to about 20 mM.

16. The pharmaceutical composition of claim 1, wherein the formulation comprises the cyclodextrin.

17. The pharmaceutical composition of claim 16, wherein the cyclodextrin is 2-hydroxypropyl-β-cyclodextrin.

18. The pharmaceutical composition of claim 16, wherein the cyclodextrin is present in an amount of from about 0.5 mM to about 200 mM.

19. The pharmaceutical composition of claim 18, wherein the cyclodextrin is present in an amount of from about 10 mM to about 50 mM.

20. The pharmaceutical composition of claim 1, wherein the composition comprises both the divalent salt and the cyclodextrin.

21. The pharmaceutical composition of claim 20, wherein the cation of the divalent salt is selected from the group consisting of Ca²⁺, Mg²⁺, Zn²⁺ and mixtures thereof and the cyclodextrin is 2-hydroxypropyl-β-cyclodextrin.

22. The pharmaceutical composition of claim 20, wherein the divalent salt is present in an amount of from about 1 mM to about 20 mM.

23. The pharmaceutical composition of claim 20, wherein the cyclodextrin is present in an amount of from about 0.5 mM to about 200 mM.

24. The pharmaceutical composition of claim 1 further comprising one or more pharmaceutically acceptable excipients.

25. The pharmaceutical composition of claim 24, wherein the one or more pharmaceutically acceptable excipients comprises a preservative.

26. The pharmaceutical composition of claim 1, wherein the composition is preservative free.

27. (canceled)

28. The pharmaceutical composition of claim 1, wherein the composition retains about 94% or more of an initial concentration of dalbavancin or pharmaceutically acceptable salt thereof after storage at when stored at about 5° C. for at least 6 months.

29. The pharmaceutical composition of claim 1, wherein the composition retains about 85% or more of an initial concentration of a B₀ component of dalbavancin or pharmaceutically acceptable salt thereof after storage at when stored at about 5° C. for at least 6 months.

30. The pharmaceutical composition of claim 1, wherein the composition is stable for at least 3 months when stored at about 25° C. and about 60% relative humidity.

31. The pharmaceutical composition of claim 1, wherein the composition is stable for at least 1 month when stored at about 40° C. and about 75% relative humidity.

32. The pharmaceutical composition of claim 1, comprising a concentration of dalbavancin or pharmaceutically acceptable salt thereof of between about 0.01 mg/ml and about 30 mg/mL.

33. The pharmaceutical composition of claim 1, wherein the composition retains about 90% or more of an initial concentration of dalbavancin or pharmaceutically acceptable salt thereof after storage at when stored at about 25° C. and about 60% relative humidity for at least 6 months.

34. The pharmaceutical composition of claim 1, wherein the composition retains about 85% or more of an initial concentration of a B₀ component of dalbavancin or pharmaceutically acceptable salt thereof after storage at when stored at about 25° C. and about 60% relative humidity for at least 3 months.

35. The pharmaceutical composition of claim 1, wherein the composition retains about 90% or more of an initial concentration of dalbavancin or pharmaceutically acceptable salt thereof after storage at when stored at about 40° C. and about 75% relative humidity for at least 1 month.

36. The pharmaceutical composition of claim 1, wherein the composition retains about 85% or more of an initial concentration of a B₀ component of dalbavancin or pharmaceutically acceptable salt thereof after storage at when stored at about 40° C. and about 75% relative humidity for at least 1 month.

37. The pharmaceutical composition of claim 1, wherein the pharmaceutical composition contains not more than about 7.0% of a MAG impurity after storage at about 5° C. for at least 6 months.

38. The pharmaceutical composition of claim 37, wherein the pharmaceutical composition contains not more than about 5.0% of a MAG impurity after storage at about 5° C. for at least 6 months.

39.-43. (canceled)

44. An aqueous pharmaceutical composition for intravenous administration consisting essentially of:
i) between about 1.0 mg/mL and about 30.0 mg/mL dalbavancin or a pharmaceutically acceptable salt thereof;
ii) a pharmaceutically acceptable buffer having a pH of from about 4.0 to about 8.0; and
iii) one or more of a pharmaceutically acceptable divalent salt and/or a pharmaceutically acceptable cyclodextrin, wherein the divalent salt is distinct from the buffer, and
wherein the pharmaceutical composition retains about 90% or more of the initial concentration of dalbavancin or pharmaceutically acceptable salt thereof after storage for at least six months when stored at about 5° C.

45. An aqueous pharmaceutical composition for intravenous administration consisting essentially of:
iv) between about 1.0 mg/mL and about 30.0 mg/mL dalbavancin or a pharmaceutically acceptable salt thereof;
v) an acetate buffer having a pH of from about 4.0 to about 5.0; and
vi) one or more of a pharmaceutically acceptable divalent salt and/or a pharmaceutically acceptable cyclodextrin, wherein the divalent salt is distinct from the buffer, and
wherein the pharmaceutical composition retains about 90% or more of the initial concentration of dalbavancin or pharmaceutically acceptable salt thereof after storage for at least six months when stored at about 5° C.

46.-56. (canceled)
